SEQUENCE LISTING (3 sequences)

SEQ ID NO: 1 - protein, 320 aa, Staphylococcus aureus
MGTEIDFDIAIIGAGPAGMTAAVYASRANLKTVMIERGIPGGQMANTEEVENFPGFEMITGPDLSTKMFEHAKKFGAVYQYGDIKSVEDKGEYKVINFGNKELTAKAVIIATGAEYKKIGVPGEQELGGRGVSYCAVCDGAFFKNKRLFVIGGGDSAVEEGTFTTKFADKVTIVHRRDELRAQRILQDRAFKNDKIDFIWSHTTKSINEKDGKVGSVTLTSTKDGSEETHEADGVFIYIGMKPLTAPFKDLGITNDVGYIVTKDDMTTSVPGIFAAGDVRDKGLRQIVTATGDGSIAAQSAAEYIEHLNDQARSHHHHHH

SEQ ID NO: 2 - protein, 320 aa, Escherichia coli
GTTKHSKLLILGSGPAGYTAAVYAARANLQPVLITGMEKGGQLTTTTEVENWPGDPNDLTGPLLMERMHEHATKFETEIIFDHINKVDLQNRPFRLNGDNGEYTCDALIIATGASARYLGLPSEEAFKGRGVSACATCDGFFYRNQKVAVIGGGNTAVEEALYLSNIASEVHLIHRRDGFRAEKILIKRLMDKVENGNIILHTNRTTEEVTGDQMGVTGVRLRDTQNSDNIESLDVAGLFVAIGHSPNTAIFEGQLELENGYIKVQSGIHGNATQTSIPGVFAAGDVMDHIYRQAITSAGTGCMAALDAERYLDGLADAK

SEQ ID NO: 3 - protein, 333 aa, Arabidopsis thaliana
MNGLETHNTRLCIVGSGPAAHTAAIYAARAELKPLLFEGWMANDIAPGGQLTTTTDVENFPGFPEGILGVELTDKFRKQSERFGTTIFTETVTKVDFSSKPFKLFTDSKAILADAVILAIGAVAKRLSFVGSGEVLGGFWNRGISACAVCDGAAPIFRNKPLAVIGGGDSAMEEANFLTKYGSKVYIIHRRDAFRASKIMQQRALSNPKIDVIWNSSVVEAYGDGERDVLGGLKVKNVVTGDVSDLKVSGLFFAIGHEPATKFLDGGVELDSDGYVVTKPGTTQTSVPGVFAAGDVQDKKYRQAITAAGTGCMAALDAEHYLQEIGSQEGKSD

(19) United States
(12) Patent Application Publication
(10) Pub. No.: US 2003/0166843 A1
(43) Pub. Date: Sep. 4, 2003

(54) Crystallization and structure determination of staphylococcus aureus thioredoxin reductase

(76) Inventor: Timothy E. Benson, Kalamazoo, MI (US)

Correspondence Address: MUETING, RAASCH & GEBHARDT, P.A., P.O. BOX 581415, MINNEAPOLIS, MN, 55458, US

(21) Appl. No.: 09/825,212

(22) Filed: Apr. 3, 2001

Related U.S. Application Data
(60) Provisional application No. 60/195,055, filed on Apr. 6, 2000.

(51) Int. Cl. C07K 1/00; C07K 14/00; C07K 17/00

(52) U.S. Cl. 530/350

(57) ABSTRACT

An unliganded form of Staphylococcus aureus thioredoxin reductase (S. aureus TrxB) has been crystallized, and the three dimensional x-ray crystal structure has been solved to 2.3 Å resolution. The x-ray crystal structure is useful for solving the structure of other molecules or molecular complexes, and designing inhibitors of S. aureus thioredoxin reductase.

This application claims the benefit of U.S. Provisional Application Serial No. 60/195,055, filed Apr. 6, 2000, which is incorporated herein by reference in its entirety.

This application incorporates by reference the material contained on the duplicate (2) compact discs submitted herewith. Each disc contains the following files:

| Name | Size | Contents | Date of File Creation |
|---|---|---|---|
| table_1.txt | 374 KB | Table 1 | Mar. 27, 2001 |

### FIELD OF THE INVENTION

The invention relates to the crystallization and structure determination of thioredoxin reductase from Staphylococcus aureus.

### BACKGROUND OF THE INVENTION

Maintaining the correct redox state of cytoplasmic enzymes is critical to cell survival. Five proteins are normally involved in the process for Escherichia coli—glutathione reductase, glutathione, glutaredoxin, thioredoxin reductase, and thioredoxin. These redundant systems provide a mechanism for the cycling of reductive enzymes during the cell cycle (i.e. ribonucleotide reductase) to their starting reduced state. Staphylococcus aureus, in contrast to E. coli, has the thioredoxin reductase/thioredoxin system as well as a Coenzyme A disulfide reductase/CoA system in place for the maintenance of the proper redox state of cellular enzymes. Since the maintenance of the correct redox state is essential to biochemical integrity, the thioredoxin reductase/thioredoxin system provides an intriguing potential for antibacterial drug discovery in S. aureus.

Extensive studies on the mechanism and kinetics of the E. coli thioredoxin reductase have been conducted and reported. The high sequence similarity between the E. coli and S. aureus species of thioredoxin reductase suggests that they conduct catalysis in a similar manner. The transfer of electrons occurs from NADPH to the bound FAD cofactor, from the FADH₂ cofactor to the Cys 135-Cys 138 disulfide, and then from the reduced cysteines to the dithiol of thioredoxin (FIG. 1). Thioredoxin reductase (which exists as a homodimer) is a two domain protein comprised of a FAD binding domain and a NADPH binding domain. The NADPH binding site includes the active site cysteines, Cys 135 and Cys 138. A third binding surface to which the other substrate thioredoxin must bind has been proposed to be created by a rotation of the NADPH binding domain. To date, only the thioredoxin reductase-FAD-Cys-Cys form of the enzyme from E. coli and Arabidopsis thaliana have been characterized structurally, although a model for the NADPH bound form of the enzyme has been proposed (Kuriyan et al., Nature, 352:172-74 (1991); Waksman et al., J. Mol. Biol., 236:800-16 (1994); and Dai et al, J. Mol. Biol., 264: 1044-57 (1994)). A very recent report describes the X-ray crystal structure of the FADH₂ form of the E. coli thioredoxin reductase and shows the flavin adopting a 34° butterfly conformation (Lennon et al., Protein Science, 8:2366-79 (1999)).

### SUMMARY OF THE INVENTION

In one aspect, the present invention provides a method for crystallizing an S. aureus thioredoxin reductase molecule or molecular complex that includes preparing purified S. aureus thioredoxin reductase at a concentration of about 1 mg/ml to about 50 mg/ml and crystallizing S. aureus thioredoxin reductase from a solution at a pH of about 6 to about 10 and comprising about 0 wt. % to about 40 wt. % DMSO and about 100 mM to about 6 M sodium formate.

In another aspect, the present invention provides crystalline forms of an S. aureus thioredoxin reductase molecule. In one embodiment, a crystal of an S. aureus thioredoxin reductase is provided having the tetragonal space group symmetry P4₃2₁2.

In another aspect, the present invention provides a scalable three dimensional configuration of points derived from structure coordinates of at least a portion of an S. aureus thioredoxin reductase molecule or molecular complex. In one embodiment, the scalable three dimensional set of points is derived from structure coordinates of at least the backbone atoms of the amino acids representing an FAD binding site and/or an NADPH binding site of an S. aureus thioredoxin reductase molecule or molecular complex. In another embodiment, the scalable three dimensional set of points is derived from structure coordinates of at least a portion of a molecule or a molecular complex that is structurally homologous to an S. aureus thioredoxin reductase molecule or molecular complex. On a molecular scale, the configuration of points derived from a homologous molecule or molecular complex have a root mean square deviation of less than about 1.4 Å, preferably less than about 1.0 Å, more preferably less than about 0.5 Å, from the structure coordinates of the molecule or complex

In another aspect, the present invention provides a molecule or molecular complex that includes at least a portion of an FAD binding site and/or an NADPH binding site of S. aureus thioredoxin reductase. In one embodiment, the S. aureus thioredoxin reductase FAD binding site includes the amino acids listed in Table 2, preferably the amino acids listed in Table 3, and more preferably the amino acids listed in Table 4, the FAD binding site being defined by a set of points having a root mean square deviation of less than about 1.1 Å from points representing the backbone atoms of the amino acids. In another embodiment, the NADPH binding site of S. aureus thioredoxin reductase includes the active site cysteines (i.e., Cys 135 and Cys 138) and the amino acids listed in Table 5, preferably the amino acids listed in Table 6, and more preferably the amino acids listed in Table 7, the binding site being defined by a set of points having a root mean square deviation of less than about 0.8 Å from points representing the backbone atoms of the amino acids.

In another aspect, the present invention provides molecules or molecular complexes that are structurally homologous to an S. aureus thioredoxin reductase molecule or molecular complex.

In another aspect, the present invention provides a machine readable storage medium including the structure coordinates of all or a portion of an S. aureus thioredoxin reductase molecule, molecular complex, a structurally homologous molecule or complex, including structurally equivalent structures as defined herein, particularly including an FAD binding site, an NADPH binding site, or similarly shaped homologous binding sites. A storage medium encoded with these data is capable of displaying on a computer screen, or similar viewing device, a three-dimensional graphical representation of a molecule or molecular complex which comprises a binding site or a similarly shaped homologous binding site.

In another aspect, the present invention provides a method for identifying inhibitors, ligands, and the like of an S. aureus thioredoxin reductase molecule by providing the coordinates of a molecule of S. aureus thioredoxin reductase to a computerized modeling system; identifying chemical entities that are likely to bind to or interfere with the molecule (e.g., screening a small molecule library); and, optionally, procuring or synthesizing and assaying the compounds or analogues derived therefrom for bioactivity. In another aspect, the present invention provides methods for designing inhibitors, ligands, and the like by providing the coordinates of a molecule of S. aureus thioredoxin reductase to a computerized modeling system; designing a chemical entity that is likely to bind to or interfere with the molecule; and, optionally, synthesizing the chemical entity and assaying the chemical entity for bioactivity. In another aspect, the present invention provides inhibitors and ligands designed by the above method. In one embodiment, a composition is provided that includes an inhibitor or ligand designed or identified by the above method. In another embodiment, the composition is a pharmaceutical composition.

In another aspect, the present invention provides a method involving molecular replacement to obtain structural information about a molecule or molecular complex of unknown structure. The method includes crystallizing the molecule or molecular complex, generating an x-ray diffraction pattern from the crystallized molecule or molecular complex, and applying at least a portion of the structure coordinates set forth in Table 1 to the x-ray diffraction pattern to generate a three-dimensional electron density map of at least a portion of the molecule or molecular complex.

In another aspect, the present invention provides a method for homology modeling an S. aureus thioredoxin reductase homolog.

Table 1 lists the atomic structure coordinates for molecules of S. aureus thioredoxin reductase as derived by x-ray diffraction from a crystal of that complex. Column 2 lists a number for the atom in the structure. Column 3 lists the element whose coordinates are measured. The first letter in the column defines the element. Column 4 lists the type of amino acid. Column 5 lists a number for the amino acid in the structure. Columns 6-8 list the crystallographic coordinates X, Y, and Z respectively. The crystallographic coordinates define the atomic position of the element measured. Column 9 lists an occupancy factor that refers to the fraction of the molecules in which each atom occupies the position specified by the coordinates. A value of “1” indicates that each atom has the same conformation, i.e., the same position, in all molecules of the crystal. Column 10 lists a thermal factor “B” that measures movement of the atom around its atomic center.

TABLE 2
Residues within about 4Å of the FAD binding site of S. aureus
thioredoxin reductase.

| | | | |
|---|---|---|---|
| ILE 12 | ILE 39 | ILE 84 | ASP 278 |
| GLY 13 | GLY 41 | ALA 111 | VAL 279 |
| ALA 14 | GLY 42 | THR 112 | ARG 285 |
| GLY 15 | GLN 43 | GLY 113 | GLN 286 |
| PRO 16 | MET 44 | ALA 114 | ILE 287 |
| ALA 17 | THR 47 | TYR 116 | VAL 288 |
| GLY 18 | VAL 50 | CYS 138 | ALA 290 |
| ILE 35 | GLU 51 | LEU 244 | TYR 1024* |
| GLU 36 | ASN 52 | PRO 247 | |
| ARG 37 | GLY 82 | PHE 248 | |
| GLY 38 | ASP 83 | GLY 277 | |

*Residues numbered greater than 1000 are from the second protein molecule present in the dimer.

TABLE 3
Residues within about 7Å of the FAD binding site of S. aureus
thioredoxin reductase.

| | | | |
|---|---|---|---|
| ILE 12 | ILE 39 | ILE 84 | ASP 278 |
| GLY 13 | GLY 41 | ALA 111 | VAL 279 |
| ALA 14 | GLY 42 | THR 112 | ARG 285 |
| GLY 15 | GLN 43 | GLY 113 | GLN 286 |
| PRO 16 | MET 44 | ALA 114 | ILE 287 |
| ALA 17 | THR 47 | TYR 116 | VAL 288 |
| GLY 18 | VAL 50 | CYS 138 | ALA 290 |
| ILE 35 | GLU 51 | LEU 244 | TYR 1024* |
| GLU 36 | ASN 52 | PRO 247 | |
| ARG 37 | GLY 82 | PHE 248 | |
| GLY 38 | ASP 83 | GLY 277 | |

*Residues numbered greater than 1000 are from the second protein molecule present in the dimer.

TABLE 4
Residues within about 10Å of the FAD binding site of S. aureus
thioredoxin reductase.

| | | | |
|---|---|---|---|
| ALA 10 | PRO 54 | LYS 117 | ILE 260 |
| ILE 11 | PHE 56 | LYS 118 | MET 266 |
| ILE 12 | GLU 57 | GLU 124 | ALA 275 |
| GLY 13 | MET 58 | GLN 125 | ALA 276 |
| ALA 14 | ILE 59 | LEU 127 | GLY 277 |
| GLY 15 | THR 60 | GLY 128 | ASP 278 |
| PRO 16 | GLY 61 | GLY 129 | VAL 279 |
| ALA 17 | PRO 62 | ARG 130 | ARG 280 |
| GLY 18 | LEU 64 | GLY 131 | LYS 282 |
| MET 19 | SER 65 | VAL 132 | GLY 283 |
| THR 20 | MET 68 | SER 133 | LEU 284 |
| ALA 21 | PHE 69 | TYR 134 | ARG 285 |
| ALA 22 | TYR 79 | CYS 135 | GLN 286 |
| VAL 33 | GLN 80 | ALA 136 | ILE 287 |
| MET 34 | TYR 81 | VAL 137 | VAL 288 |
| ILE 35 | GLY 82 | CYS 138 | THR 289 |
| GLU 36 | ASP 83 | ASP 139 | ALA 290 |
| ARG 37 | ILE 84 | GLY 140 | THR 291 |
| GLY 38 | LYS 85 | ALA 141 | GLY 292 |
| ILE 39 | SER 86 | PHE 142 | ASP 293 |
| PRO 40 | VAL 87 | PHE 143 | GLY 294 |
| GLY 41 | ILE 96 | PHE 236 | SER 295 |
| GLY 42 | ASN 97 | ILE 237 | ALA 297 |
| GLN 43 | PHE 98 | GLY 240 | THR 1020* |
| MET 44 | GLY 99 | MET 241 | VAL 1023* |
| ALA 45 | VAL 108 | LYS 242 | TYR 1024* |
| ASN 46 | ILE 109 | PRO 243 | ARG 1027* |
| THR 47 | ILE 110 | LEU 244 | PRO 1054* |
| GLU 48 | ALA 111 | THR 245 | HIS 1071* |
| GLU 49 | THR 112 | ALA 246 | PHE 1075* |
| VAL 50 | GLY 113 | PRO 247 | THR 1291* |
| GLU 51 | ALA 114 | PHE 248 | GLY 1292* |
| ASN 52 | GLU 115 | LYS 249 | SER 1295* |
| PHE 53 | TYR 116 | TYR 259 | GLN 1299* |

*Residues numbered greater than 1000 are from the second protein molecule present in the dimer.

TABLE 5
Residues within about 4Å of Cys135-Cys 138 at the active site.

| | | | |
|---|---|---|---|
| ASN 52 | ALA 136 | GLU 160 | FAD 501 |
| TYR 116 | VAL 137 | PHE 236 | ARG 1027* |
| SER 133 | ASP 139 | TYR 238 | |
| TYR 134 | GLY 140 | GLN 286 | |

*Residues numbered greater than 1000 are from the second protein molecule present in the dimer.

TABLE 6
Residues within about 7Å of Cys135-Cys 138 at the active site.

| | | | |
|---|---|---|---|
| GLN 43 | TYR 134 | GLU 160 | ARG 285 |
| THR 47 | ALA 136 | PHE 163 | GLN 286 |
| GLU 49 | VAL 137 | LEU 164 | ILE 287 |
| VAL 50 | ASP 139 | VAL 235 | FAD 501 |
| GLU 51 | GLY 140 | PHE 236 | TYR 1024* |
| ASN 52 | ALA 141 | ILE 237 | ARG 1027* |
| TYR 116 | PHE 142 | TYR 238 | |
| VAL 132 | PHE 143 | MET 241 | |
| SER 133 | SER 156 | LEU 284 | |

*Residues numbered greater than 1000 are from the second protein molecule present in the dimer.

TABLE 7
Residues within about 10Å of Cys135-Cys 138 at the active site.

| | | | |
|---|---|---|---|
| GLN 43 | GLY 128 | SER 156 | GLY 283 |
| MET 44 | GLY 129 | ALA 157 | LEU 284 |
| ASN 46 | GLY 131 | GLU 159 | ARG 285 |
| THR 47 | VAL 132 | GLU 160 | GLN 286 |
| GLU 48 | SER 133 | GLY 161 | ILE 287 |
| GLU 49 | TYR 134 | PHE 163 | VAL 288 |
| VAL 50 | ALA 136 | LEU 164 | THR 289 |
| GLU 51 | VAL 137 | PHE 167 | FAD 501 |
| ASN 52 | ASP 139 | GLY 234 | VAL 1023* |
| PHE 53 | GLY 140 | VAL 235 | TYR 1024* |
| MET 58 | ALA 141 | PHE 236 | ARG 1027* |
| GLY 61 | PHE 142 | ILE 237 | ALA 1028* |
| ALA 114 | PHE 143 | TYR 238 | LYS 1074* |
| GLU 115 | LEU 148 | ILE 239 | PHE 1075* |
| TYR 116 | PHE 149 | GLY 240 | SER 1295* |
| LYS 117 | VAL 150 | MET 241 | GLN 1299* |
| LYS 118 | ILE 151 | ASP 278 | |

*Residues numbered greater than 1000 are from the second protein molecule present in the dimer.

Definitions

Two data sets are considered isomorphous if, after scaling,

$$\frac{Δ F}{F} = \frac{\sum \vert {F}_{1} - {F}_{2} \vert}{\sum {F}_{1}}$$

is less than about 35% for the reflections between 8 Å and 4 Å, where F is a structure factor.

Abbreviations

The following abbreviations are used throughout this disclosure:

Staphylococcus aureus (S. aureus)

Escherichia coli (E. coli)

Arabidopsis thaliana (A. thaliana)

thioredoxin reductase (TrxB)

Flavin adenine dinucleotide (FAD)

Oxidized flavin adenine dinucleotide (FADₒₓ)

Reduced flavin adenine dinucleotide (FADH₂ or FAD_{red})

β-nicotinamide adenine dinucleotide phosphate (NADP)

Oxidized β-nicotinamide adenine dinucleotide phosphate (NADP+)

Reduced β-nicotinamide adenine dinucleotide phosphate (NADPH)

Isopropylthio-β-D-galactoside (IPTG).

Dimethyl sulfoxide (DMSO)

Polyethylene glycol (PEG)

Multiple anomalous dispersion (MAD)

Root mean square (r.m.s.)

Root mean square deviation (r.m.s.d.)

The following amino acid abbreviations are used throughout this disclosure:

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

### BRIEF DESCRIPTION OF THE FIGURES

FIG. 1 is a schematic illustration of a redox cycle catalyzed by thioredoxin reductase and thioredoxin.

FIG. 2 illustrates anomalous difference Patterson Maps for Harker sections u=½ and v=½. FIGS. 2a and 2b are the Patterson Maps for two different data sets.

FIG. 3 illustrates anomalous difference Patterson Maps for Harker section w=½. FIGS. 3a and 3b are the Patterson Maps for two different data sets.

FIG. 4 illustrates anomalous difference Patterson Maps for Patterson section w=0. FIGS. 3a and 3b are the Patterson Maps for two different data sets. The peaks in this Patterson map section result from the two fold non-crystallographic symmetry relating the two monomers in the asymmetric unit.

FIG. 5 illustrates electron density maps from multiple anomalous dispersion phases with a portion of the final model refined against the second data set. FIGS. 5a and 5b illustrate electron density maps for the first data set before solvent flattening (FIG. 5a) and after solvent flattening (FIG. 5b). FIGS. 5c and 5d illustrate electron density maps for the second data set before solvent flattening (FIG. 5c) and after solvent flattening (FIG. 5d). FIG. 6 illustrates electron density maps from multiple anomalous dispersion phases showing a helix. FIGS. 6a and 6b illustrate electron density maps for the first data set before solvent flattening (FIG. 6a) and after solvent flattening (FIG. 6b). FIGS. 6c and 6d illustrate electron density maps for the second data set before solvent flattening (FIG. 6c) and after solvent flattening (FIG. 6d). While both maps clearly show the secondary structure, the maps illustrated in FIGS. 6c and 6d are clearer.

FIG. 7 illustrates the structure of the S. aureus thioredoxin reductase dimer. One monomer is dark gray and the other is medium gray. The flavin cofactors are light gray.

FIG. 8 illustrates electron density maps near the active site showing the flavin cofactor and the disulfide bond between Cys 135 and Cys 138. FIG. 8a illustrates a solvent flattened multiple anomalous dispersion map. FIG. 8b illustrates the final 2Fo-Fc electron density map after refinement.

FIG. 9 is a schematic illustration of binding interactions between S. aureus thioredoxin reductase and the FAD cofactor.

FIG. 10 illustrates a stereoview of the superposition of all corresponding residues (r.m.s.d. 2.12 Å) between E. coli thioredoxin reductase (light residues 5-54, 59-190, 198-224, 229-255, 261-266, 274-316) and S. aureus thioredoxin reductase (dark; residues 6-55, 59-190, 195-221, 225-251, 258-263, 266-308).

FIG. 11 illustrates the sequence alignment of S. aureus thioredoxin reductase (SEQ ID NO: 1) and E. coli (SEQ ID NO: 2). Underlined blocks indicate identical residues while dark shaded blocks indicate similar residues. This alignment was originally conducted in Vector NTI.

FIG. 12 illustrates a stereoview of the superposition of residues in domain 1 (r.m.s.d. 1.47 Å) between E. coli thioredoxin reductase (light; residues 5-54, 59-116, 246-255, 261-266, 274-316) and S. aureus) thioredoxin reductase (dark; residues 6-55, 59-116, 242-251, 258-263, 266-308).

FIG. 13 illustrates a stereoview of the superposition of residues in domain 2 (r.m.s.d. 1.12 Å) between E. coli thioredoxin reductase (light; residues 117-190, 198-224, 229-245) and S. aureus) thioredoxin reductase (dark; residues 117-190, 195-221, 225-241).

FIG. 14 illustrates a stereoview of the superposition of all corresponding residues (r.m.s.d. 1.41 Å) between A. thaliana thioredoxin reductase (light; residues 6-35, 39-55, 58-124, 130-141, 145-204, 217-253, 276-316) and S. aureus) thioredoxin reductase (dark; residues 7-36, 40-56, 58-124, 130-141, 145-204, 213-249, 268-308).

FIG. 15 illustrates the sequence alignment of A. thaliana (SEQ ID NO: 3) and S. aureus thioredoxin reductase (SEQ ID NO: 1). Underlined blocks indicate identical residues while dark shaded blocks indicate similar residues.

FIG. 16 illustrates a stereoview of the superposition of domain 1 residues (r.m.s.d. 1.12 Å) between A. thaliana thioredoxin reductase (light; residues 6-35, 39-55, 58-116, 246-253, 276-316) and S. aureus) thioredoxin reductase (dark; residues 7-36, 40-56, 58-116, 242-249, 268-308).

FIG. 17 illustrates a stereoview of the superposition of domain 2 residues (r.m.s.d. 0.86 Å) between A. thaliana thioredoxin reductase (light; residues 117-124, 130-141, 145-204, 217-245) and S. aureus) thioredoxin reductase (dark; residues 117-124, 130-141, 145-204, 213-241).

FIG. 18 illustrates a secondary structure diagram for S. aureus thioredoxin reductase. Points where deletions or insertions occur with respect to the E. coli or A. thatiana structures are shown.

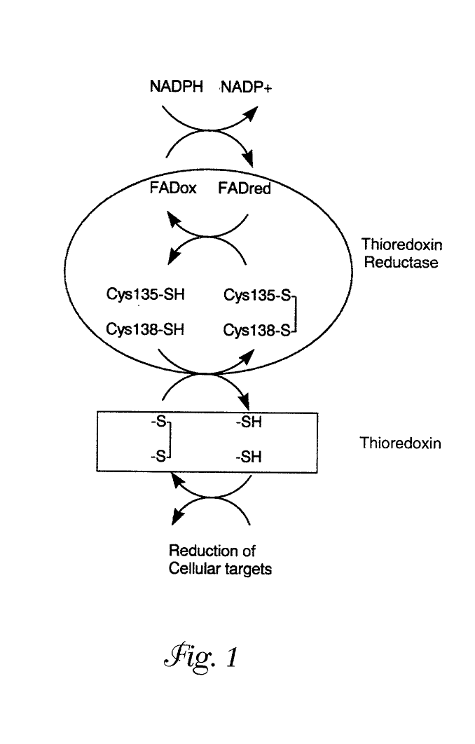

### DETAILED DESCRIPTION OF THE PREFERRED EMBODIMENTS

Crystalline Form(s) and Method of Making

Applicants have produced crystals comprising S. aureus thioredoxin reductase that are suitable for x-ray crystallographic analysis. The three-dimensional structure of S. aureus thioredoxin reductase was solved using x-ray crystallography. Preferably, the crystal has tetragonal space group symmetry P4₃2₁2. More preferably, the crystal comprises rectangular shaped unit cells, each unit cell having dimensions wherein a is about 90±20 Å, b is about 90±20 Å, and c is about 190±30 Å. The crystallized enzyme is a functional dimer having two thioredoxin reductase molecules in the asymmetric unit.

Accordingly, one embodiment of the invention provides an S. aureus thioredoxin reductase or S. aureus thioredoxin reductase/ligand crystal. Preferably, the crystals are crystallized from a concentrated sodium formate solution. More preferably the crystals are crystallized from a solution at a pH of about 6 to about 10 and comprising about 100 mM to about 6 M sodium formate. Inclusion of buffers as well as other additives such as PEG and DMSO in the crystallization solution are apparent to those skilled in the art and may result in similar crystals.

The invention further includes an S. aureus thioredoxin reductase crystal or S. aureus thioredoxin reductase/ligand crystal that is isomorphous with an S. aureus thioredoxin reductase crystal characterized by a unit cell having dimensions wherein a is about 90±20 Å, b is about 90±20 Å, c is about 190±30 Å, and α=β=γ90°.

X-Ray Crystallographic Analysis

Each of the constituent amino acids of S. aureus thioredoxin reductase is defined by a set of structure coordinates as set forth in Table 1. The term “structure coordinates” refers to Cartesian coordinates derived from mathematical equations related to the patterns obtained on diffraction of a monochromatic beam of x-rays by the atoms (scattering centers) of an S. aureus thioredoxin reductase complex in crystal form. The diffraction data are used to calculate an electron density map of the repeating unit of the crystal. The electron density maps are then used to establish the positions of the individual atoms of the S. aureus thioredoxin reductase protein or protein/ligand complex.

Slight variations in structure coordinates can be generated by mathematically manipulating the S. aureus thioredoxin reductase or S. aureus thioredoxin reductase/ligand structure coordinates. For example, the structure coordinates set forth in Table 1 could be manipulated by crystallographic permutations of the structure coordinates, fractionalization of the structure coordinates, integer additions or subtractions to sets of the structure coordinates, inversion of the structure coordinates or any combination of the above. Alternatively, modifications in the crystal structure due to mutations, additions, substitutions, and/or deletions of amino acids, or other changes in any of the components that make up the crystal, could also yield variations in structure coordinates. Such slight variations in the individual coordinates will have little effect on overall shape. If such variations are within an acceptable standard error as compared to the original coordinates, the resulting three-dimensional shape is considered to be structurally equivalent. Structural equivalence is described in more detail below.

It should be noted that slight variations in individual structure coordinates of the S. aureus thioredoxin reductase or S. aureus thioredoxin reductase/ligand complex, as defined above, would not be expected to significantly alter the nature of ligands that could associate with the binding sites. Thus, for example, a ligand that bound to a binding site of S. aureus thioredoxin reductase would also be expected to bind to or interfere with another binding site whose structure coordinates define a shape that falls within the acceptable error.

Overview of the Structure

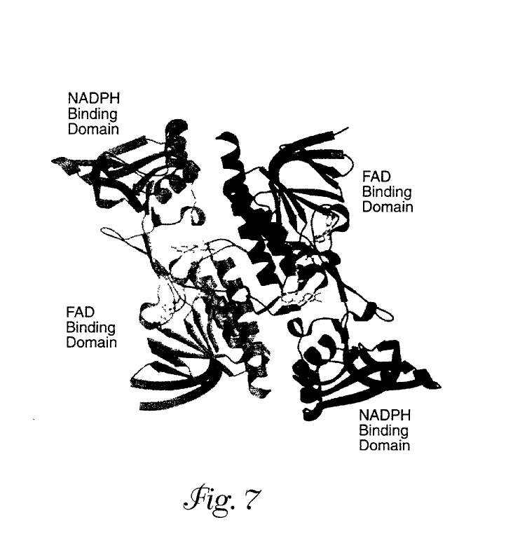

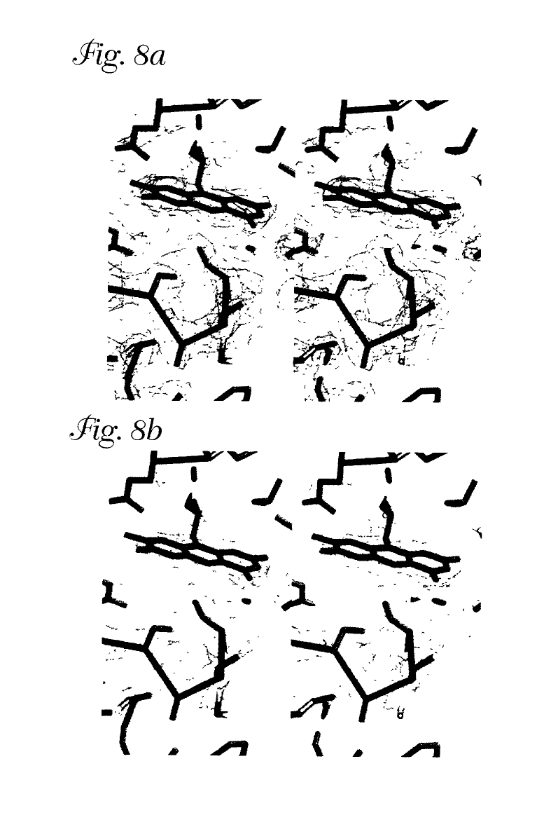

S. aureus thioredoxin reductase shows the same overall fold and domain structure as observed in the E. coli and A. thaliana thioredoxin reductases (Kuriyan et al., Nature, 352:172-74 (1991); Waksman et al., J. Mol. Biol., 236:800-16 (1994); and Dai et al, J. Mol. Biol., 264: 1044-57 (1994)). The enzyme is composed of two domains classified as the FAD binding domain and a proposed NADPH binding domain (FIG. 7). The FAD domain has a central five stranded parallel β sheet surrounded by three α helices on one side and a three stranded antiparallel β sheet on the other. The second domain, the NADPH domain, has a central four stranded parallel β sheet surrounded by two helices on one side and a three stranded antiparallel β sheet on the other. The isoalloxazine ring of the flavin cofactor rests at the interface of these two domains adjacent to the two active site cysteines, Cys135 and Cys 138, which are involved in the redox cycle with the protein substrate thioredoxin. In this crystal structure, the two cysteines form a disulfide bond as indicated by the electron density map (FIG. 8). The presence of two active site cysteines will necessitate vigilance to ensure compounds identified by high throughput screening are not acting by electrophilic substitution to these residues.

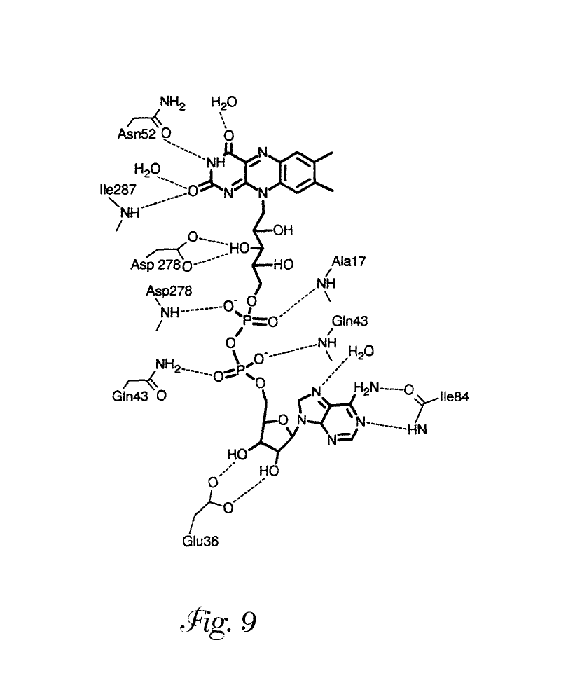

The bound flavin cofactor interacts with the protein by a series of residues similar to those observed in the E. coli and A. thaliana thioredoxin reductases. There are no apparent residues that interact with N1 of the FAD. Most of the interactions with the isoalloxazine ring are conserved side chain or main chain interactions. The most notable difference is the interaction between Glu36 at the two ribose hydroxyl groups which is not present in either the E. coli (Ser13 interacts with only one of the hydroxyl groups) or A. thaliana (no side chain or main chain interactions are observed). A detailed diagram of all the main chain and side chain interactions from the S. aureus protein and the FAD cofactor is shown in FIG. 9. There do appear to be several small pockets that an inhibitor of thioredoxin reductase might take advantage of during binding. Yet, it is possible that the enzyme would adapt to some other conformation in the presence of a ligand, since the N1 of the isoalloxazine ring of the flavin and the two active site cysteines (the two points for oxidation and reduction) are not accessible in the present conformation. Alternatively, a suitable inhibitor might be found which locks the S. aureus thioredoxin reductase into its current conformation making it unable to transfer electrons from NADPH or to thioredoxin.

Comparison to other Thioredoxin Reductase Structures

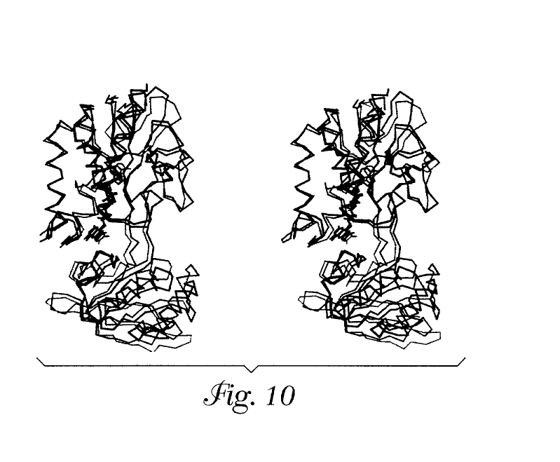

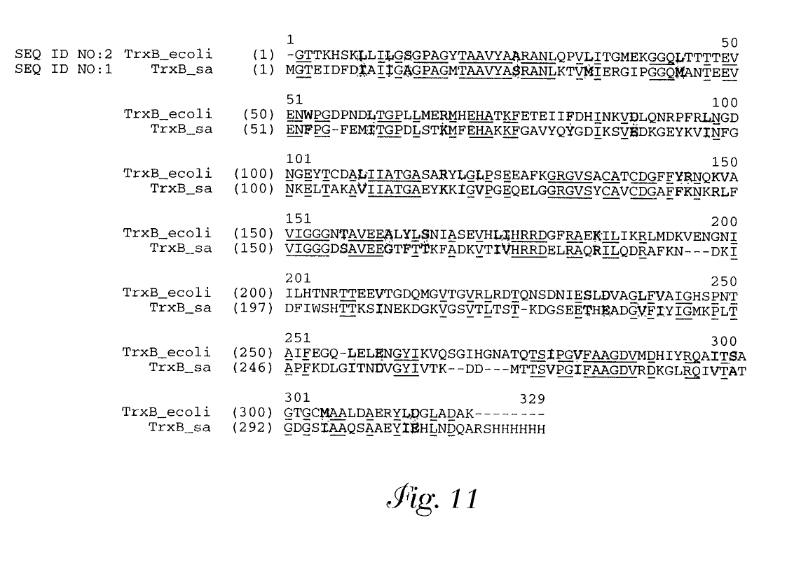

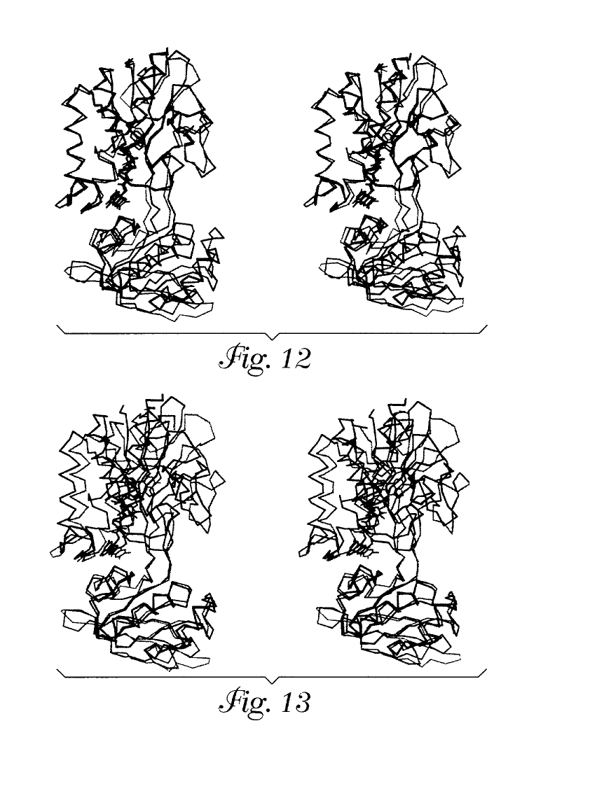

A detailed comparison of the S. aureus thioredoxin reductase to its homologs from E. coli and A. thaliana shows a high level of similarity. Superposition of the C_{α} atoms for all residues found in common between S. aureus and E. coli thioredoxin reductase gave an r.m.s. deviation of 2.12 Å (FIG. 10). Sequence alignment as shown in FIG. 11 shows an identity of 40% and a similarity of 52%. The comparison of the two domains shows a high degree of similarity with an r.m.s. deviation of 1.47 Å between the FAD domains (domain 1) and an r.m.s. deviation of 1.12 Å between the NADPH domains (domain 2) (FIGS. 12-13). Alignment of domain 2 from both the S. aureus and E. coli structures reveals a concerted movement of domain 1. This motion could be a result of either crystal packing forces within the S. aureus thioredoxin reductase crystals or the fact that a dimer is present in the asymmetric unit. In both E. coli and A. thaliana thioredoxin reductase crystal forms reported to date, the active dimer is formed by crystallographic symmetry instead of the non-crystallographic symmetry found in the S. aureus crystals.

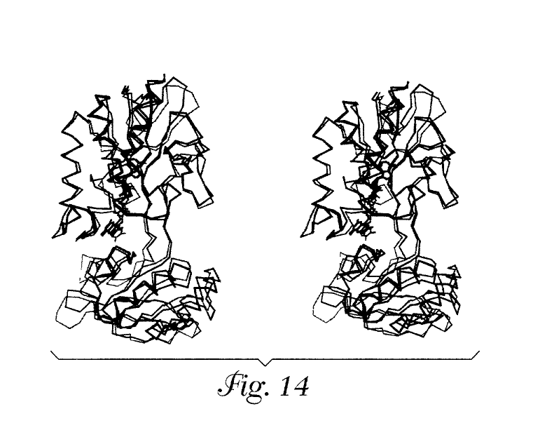

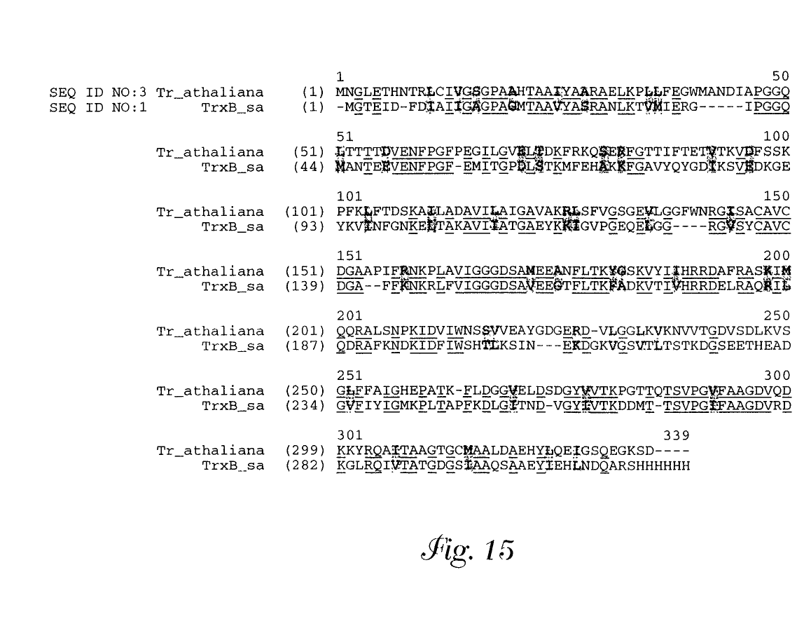

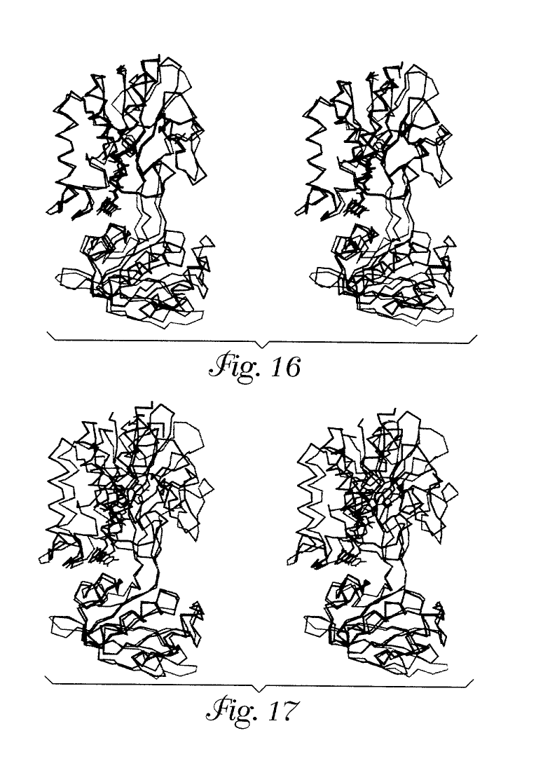

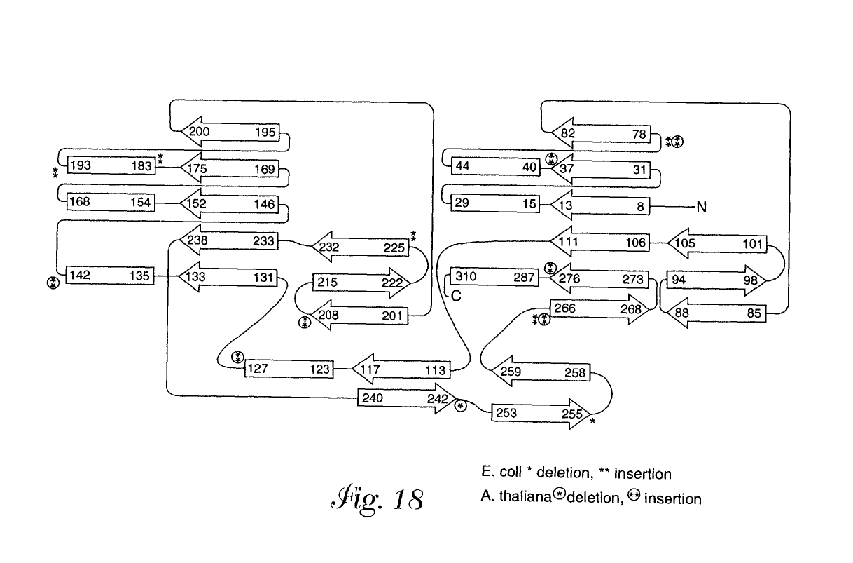

A similar comparison can be made for the S. aureus and A. thaliana thioredoxin reductase structures. Superposition of the C_{α} atoms for all residues found in common between these two structures gave an r.m.s. deviation of 1.41 Å revealing a slightly better alignment than for the E. coli comparison (FIG. 14). The lower r.m.s. deviation is a reflection of the higher sequence conservation between the S. aureus and A. thaliana thioredoxin reductase sequences (44% identical and 58% similar) (FIG. 15). A superposition of domain 1 from S. aureus and A. thaliana thioredoxin reductase showed even better alignment with an r.m.s. deviation of 1.12 Å (FIG. 16), while the superposition of domain 2 showed the highest similarity with an r.m.s. deviation of 0.86 Å (FIG. 17). A similar motion of domain 1 with respect to domain 2 can be observed in the superposition of the S. aureus and A. thaliana structures. In both structures there are several regions where insertions or deletions in the sequence effect the protein structure. The most notable insertion can be seen by comparing S. aureus thioredoxin reductase with A. thaliana thioredoxin reductase around residue 38 near the adenine of the flavin. There is a stretch of 5 residues in the A. thaliana structure which cover the adenine of the flavin in that structure which is not present in the S. aureus or E. coli structures. Points at which there are differences in the three structures are indicated in the secondary structure diagram shown in FIG. 18.

Binding Sites/Active Sites/other Structural Features

The present invention has provided, for the first time, information about the shape and structure of the binding sites and/or active sites of S. aureus thioredoxin reductase.

Binding sites are of significant utility in fields such as drug discovery. The association of natural ligands or substrates with the binding sites of their corresponding receptors or enzymes is the basis of many biological mechanisms of action. Similarly, many drugs exert their biological effects through association with the binding sites of receptors and enzymes. Such associations may occur with all or any parts of the binding site. An understanding of such associations helps lead to the design of drugs having more favorable associations with their target, and thus improved biological effects. Therefore, this information is valuable in designing potential inhibitors of S. aureus thioredoxin reductase-like binding sites, as discussed in more detail below.

The term “binding site,” as used herein, refers to a region of a molecule or molecular complex, that, as a result of its shape, favorably associates with another chemical entity or compound. Thus, a binding site may include or consist of features such as interfaces between domains. Chemical entities or compounds that may associate with a binding site includes, but is not limited to, cofactors, substrates, inhibitors, agonists, antagonists, etc.

The FAD binding site of S. aureus thioredoxin reductase is located in domain 1, and preferably includes the amino acids listed in Table 2, more preferably the amino acids listed in Table 3, and most preferably the amino acids listed in Table 4, as represented by the structure coordinates listed in Table 1. It will be readily apparent to those of skill in the art that the numbering of amino acids in other isoforms of S. aureus thioredoxin reductase may be different than that of recombinant S. aureus thioredoxin reductase expressed in E. coli. Alternatively, the FAD binding site of S. aureus thioredoxin reductase includes those amino acids whose backbone atoms are situated within about 4 Å, more preferably within about 7 Å, most preferably within about 10 Å, of one or more constituent atoms of a bound FAD cofactor or analog, as determined from the structure coordinates listed in Table 1.

The putative NADPH binding site of S. aureus thioredoxin reductase is located in domain 2 and includes Cys 135, Cys 138, and preferably the amino acids listed in Table 5, more preferably the amino acids listed in Table 6, and most preferably the amino acids listed in Table 7, as represented by the structure coordinates listed in Table 1. Alternatively, the putative NADPH binding site of S. aureus thioredoxin reductase includes those amino acids whose backbone atoms are situated within about 4 Å, more preferably within about 7 Å, most preferably within about 10 Å, of one or more constituent atoms of a bound ligand, as determined from the structure coordinates listed in Table 1.

The amino acid constituents of an S. aureus thioredoxin reductase binding site as defined herein, as well as selected constituent atoms thereof, are positioned in three dimensions in accordance with the structure coordinates listed in Table 1. In one aspect, the structure coordinates defining the binding site of S. aureus thioredoxin reductase include structure coordinates of all atoms in the constituent amino acids; in another aspect, the structure coordinates of the binding site include structure coordinates of just the backbone atoms of the constituent atoms.

The term “S. aureus thioredoxin reductase-like” binding site refers to a portion of a molecule or molecular complex whose shape is sufficiently similar to at least a portion of a binding site of S. aureus thioredoxin reductase as to be expected to bind FAD, NADPH, or a structurally related cofactor. A structurally equivalent FAD binding site is defined by a root mean square deviation from the structure coordinates of the backbone atoms of the amino acids that make up the binding site in S. aureus thioredoxin reductase (as set forth in Table 1) of at most about 1.1 Å. A structurally equivalent NADPH binding site is defined by a root mean square deviation from the structure coordinates of the backbone atoms of the amino acids that make up the putative NADPH binding site in S. aureus thioredoxin reductase (as set forth in Table 1) of at most about 0.8 Å. How this calculation is obtained is described below.

The term “associating with” refers to a condition of proximity between a chemical entity or compound, or portions thereof, and an S. aureus thioredoxin reductase molecule or portions thereof. The association may be non-covalent, wherein the juxtaposition is energetically favored by hydrogen bonding, van der Waals forces, or electrostatic interactions, or it may be covalent.

Accordingly, the invention thus provides molecules or molecular complexes comprising an S. aureus thioredoxin reductase binding site or S. aureus thioredoxin reductase-like binding site, as defined by the sets of structure coordinates described above.

Three-Dimensional Configurations

The structure coordinates generated for S. aureus thioredoxin reductase or the S. aureus thioredoxin reductase/ligand complex or one of its binding sites shown in Table 1 define a unique configuration of points in space. Those of skill in the art understand that a set of structure coordinates for protein or an protein/ligand complex, or a portion thereof, define a relative set of points that, in turn, define a configuration in three dimensions. A similar or identical configuration can be defined by an entirely different set of coordinates, provided the distances and angles between coordinates remain essentially the same. In addition, a scalable configuration of points can be defined by increasing or decreasing the distances between coordinates by a scalar factor while keeping the angles essentially the same.

The present invention thus includes the scalable three-dimensional configuration of points derived from the structure coordinates of at least a portion of an S. aureus thioredoxin reductase molecule or molecular complex, as listed in Table 1, as well as structurally equivalent configurations, as described below. Preferably, the scalable three-dimensional configuration includes points derived from structure coordinates representing the locations of a plurality of the amino acids defining an S. aureus thioredoxin reductase binding site.

In one embodiment, the scalable three-dimensional configuration includes points derived from structure coordinates representing the locations of the backbone atoms of a plurality of amino acids defining the S. aureus thioredoxin reductase FAD binding site, preferably the amino acids listed is Table 2, more preferably the amino acids listed in Table 3, and most preferably the amino acids listed in Table 4; in another embodiment, the three-dimensional configuration includes points derived from structure coordinates representing the locations of the side chain and the backbone atoms (other than hydrogens) of a plurality of the amino acids defining the S. aureus thioredoxin reductase FAD binding site, preferably the amino acids listed is Table 2, more preferably the amino acids listed in Table 3, and most preferably the amino acids listed in Table 4.

In another embodiment, the scalable three-dimensional configuration includes points derived from structure coordinates representing the locations of the backbone atoms of a plurality of amino acids defining an NADPH binding site of S. aureus thioredoxin reductase, the amino acids including Cys 135, Cys 138, and preferably the amino acids listed is Table 5, more preferably the amino acids listed in Table 6, and most preferably the amino acids listed in Table 7; in another embodiment, the three-dimensional configuration includes points derived from structure coordinates representing the locations of the side chain and the backbone atoms (other than hydrogens) of a plurality of the amino acids defining an NADPH binding site of S. aureus thioredoxin reductase, the amino acids including Cys 135, Cys 138, and preferably the amino acids listed in Table 5, more preferably the amino acids listed in Table 6, and most preferably the amino acids listed in Table 7.

Likewise, the invention also includes the three-dimensional configuration of points derived from structure coordinates of molecules or molecular complexes that are structurally homologous to S. aureus thioredoxin reductase, as well as structurally equivalent configurations. Structurally homologous molecules or molecular complexes are defined below. Advantageously, structurally homologous molecules can be identified using the structure coordinates of S. aureus thioredoxin reductase (Table 1) according to a method of the invention.

The configurations of points in space derived from structure coordinates according to the invention can be visualized as, for example, a holographic image, a stereodiagram, a model or a computer-displayed image, and the invention thus includes such images, diagrams or models.

Structurally Equivalent Crystal Structures

Various computational analyses can be used to determine whether a molecule or the binding site portion thereof is “structurally equivalent,” defined in terms of its three-dimensional structure, to all or part of S. aureus thioredoxin reductase or its binding sites. Such analyses may be carried out in current software applications, such as the Molecular Similarity application of QUANTA (Molecular Simulations Inc., San Diego, Calif.) version 4.1, and as described in the accompanying User's Guide.

The Molecular Similarity application permits comparisons between different structures, different conformations of the same structure, and different parts of the same structure. The procedure used in Molecular Similarity to compare structures is divided into four steps: (1) load the structures to be compared; (2) define the atom equivalences in these structures; (3) perform a fitting operation; and (4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention equivalent atoms are defined as protein backbone atoms (N, Cα, C, and O) for all conserved residues between the two structures being compared. A conserved residue is defined as a residue that is structurally or functionally equivalent. Only rigid fitting operations are considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this invention, any molecule or molecular complex or binding site thereof, or any portion thereof, that has a root mean square deviation of conserved residue backbone atoms (N, Cα, C, O) of less than about 1.4 Å, when superimposed on the relevant backbone atoms described by the reference structure coordinates listed in Table 1, is considered “structurally equivalent” to the reference molecule. That is to say, the crystal structures of those portions of the two molecules are substantially identical, within acceptable error. Particularly preferred structurally equivalent molecules or molecular complexes are those that are defined by the entire set of structure coordinates in Table 1, ± a root mean square deviation from the conserved backbone atoms of those amino acids of not more than 1.4 Å. More preferably, the root mean square deviation is less than about 0.8 Å.

The term “root mean square deviation” means the square root of the arithmetic mean of the squares of the deviations. It is a way to express the deviation or variation from a trend or object. For purposes of this invention, the “root mean square deviation” defines the variation in the backbone of a protein from the backbone of S. aureus thioredoxin reductase or a binding portion thereof, as defined by the structure coordinates of S. aureus thioredoxin reductase described herein.

Machine Readable Storage Media

Transformation of the structure coordinates for all or a portion of S. aureus thioredoxin reductase or the S. aureus thioredoxin reductase/ligand complex or one of its binding sites, for structurally homologous molecules as defined below, or for the structural equivalents of any of these molecules or molecular complexes as defined above, into three-dimensional graphical representations of the molecule or complex can be conveniently achieved through the use of commercially-available software.

The invention thus further provides a machine-readable storage medium comprising a data storage material encoded with machine readable data which, when using a machine programmed with instructions for using said data, is capable of displaying a graphical three-dimensional representation of any of the molecule or molecular complexes of this invention that have been described above. In a preferred embodiment, the machine-readable data storage medium comprises a data storage material encoded with machine readable data which, when using a machine programmed with instructions for using said data, is capable of displaying a graphical three-dimensional representation of a molecule or molecular complex comprising all or any parts of an S. aureus thioredoxin reductase binding site or an S. aureus thioredoxin reductase-like binding site, as defined above. In another preferred embodiment, the machine-readable data storage medium is capable of displaying a graphical three-dimensional representation of a molecule or molecular complex defined by the structure coordinates of all of the amino acids in Table 1, ± a root mean square deviation from the backbone atoms of said amino acids of not more than 1.4 Å.

In an alternative embodiment, the machine-readable data storage medium comprises a data storage material encoded with a first set of machine readable data which comprises the Fourier transform of the structure coordinates set forth in Table 1, and which, when using a machine programmed with instructions for using said data, can be combined with a second set of machine readable data comprising the x-ray diffraction pattern of a molecule or molecular complex to determine at least a portion of the structure coordinates corresponding to the second set of machine readable data.

For example, a system for reading a data storage medium may include a computer comprising a central processing unit (“CPU”), a working memory which may be, e.g., RAM (random access memory) or “core” memory, mass storage memory (such as one or more disk drives or CD-ROM drives), one or more display devices (e.g., cathode-ray tube (“CRT”) displays, light emitting diode (“LED”) displays, liquid cyrstal displays (“LCDs”), electroluminescent displays, vacuum fluorescent displays, field emission displays (“FEDs”), plasma displays, projection panels, etc.), one or more user input devices (e.g., keyboards, microphones, mice, touch screens, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as consumer electronics and appliances.

Input hardware may be coupled to the computer by input lines and may be implemented in a variety of ways. Machine-readable data of this invention may be inputted via the use of a modem or modems connected by a telephone line or dedicated data line. Alternatively or additionally, the input hardware may comprise CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of a binding site of this invention using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of this invention. Such programs are discussed in reference to the computational methods of drug discovery as described herein. References to components of the hardware system are included as appropriate throughout the following description of the data storage medium.

Machine-readable storage devices useful in the present invention include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data..

Structurally Homologous Molecules, Molecular Complexes, and Crystal Structures

The structure coordinates set forth in Table 1 can be used to aid in obtaining structural information about another crystallized molecule or molecular complex. A “molecular complex” means a protein in covalent or non-covalent association with a chemical entity or compound. The method of the invention allows determination of at least a portion of the three-dimensional structure of molecules or molecular complexes which contain one or more structural features that are similar to structural features of S. aureus thioredoxin reductase. These molecules are referred to herein as “structurally homologous” to S. aureus thioredoxin reductase. Similar structural features can include, for example, regions of amino acid identity, conserved active site or binding site motifs, and similarly arranged secondary structural elements (e.g., α helices and β sheets). Optionally, structural homology is determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Preferably, two amino acid sequences are compared using the Blastp program, version 2.0.9, of the BLAST 2 search algorithm, as described by Tatusova et al., FEMS Microbiol Lett., 174, 247-50 (1999), and available at http://www.ncbi.nlm.nih.gov/gorf/bl2.html. Preferably, the default values for all BLAST 2 search parameters are used, including matrix=BLOSUM62; open gap penalty=11, extension gap penalty=1, gap×_dropoff=50, expect=10, wordsize=3, and filter on. In the comparison of two amino acid sequences using the BLAST search algorithm, structural similarity is referred to as “identity.” Preferably, a structurally homologous molecule is a protein that has an amino acid sequence sharing at least 65% identity with a native or recombinant amino acid sequence of S. aureus thioredoxin reductase (e.g., SEQ ID NO: 1). More preferably, a protein that is structurally homologous to S. aureus thioredoxin reductase includes at least one contiguous stretch of at least 50 amino acids that shares at least 80% amino acid sequence identity with the analogous portion of the native or recombinant S. aureus thioredoxin reductase (e.g., SEQ ID NO: 1). Methods for generating structural information about the structurally homologous molecule or molecular complex are well-known and include, for example, molecular replacement techniques.

Therefore, in another embodiment this invention provides a method of utilizing molecular replacement to obtain structural information about a molecule or molecular complex whose structure is unknown comprising the steps of:

(a) crystallizing the molecule or molecular complex of unknown structure;

(b) generating an x-ray diffraction pattern from said crystallized molecule or molecular complex; and

(c) applying at least a portion of the structure coordinates set forth in Table 1 to the x-ray diffraction pattern to generate a three-dimensional electron density map of the molecule or molecular complex whose structure is unknown.

By using molecular replacement, all or part of the structure coordinates of S. aureus thioredoxin reductase or the S. aureus thioredoxin reductase/ligand complex as provided by this invention (and set forth in Table 1) can be used to determine the structure of a crystallized molecule or molecular complex whose structure is unknown more quickly and efficiently than attempting to determine such information ab initio.

Molecular replacement provides an accurate estimation of the phases for an unknown structure. Phases are a factor in equations used to solve crystal structures that cannot be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a structurally homologous portion has been solved, the phases from the known structure provide a satisfactory estimate of the phases for the unknown structure.

Thus, this method involves generating a preliminary model of a molecule or molecular complex whose structure coordinates are unknown, by orienting and positioning the relevant portion of S. aureus thioredoxin reductase or the S. aureus thioredoxin reductase/ligand complex according to Table 1 within the unit cell of the crystal of the unknown molecule or molecular complex so as best to account for the observed x-ray diffraction pattern of the crystal of the molecule or molecular complex whose structure is unknown. Phases can then be calculated from this model and combined with the observed x-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex (E. Lattman, “Use of the Rotation and Translation Functions,” in Meth. Enzymol., 115, pp. 55-77 (1985); M. G. Rossman, ed., “The Molecular Replacement Method,” Int. Sci. Rev. Ser., No. 13, Gordon & Breach, New York (1972)).

Structural information about a portion of any crystallized molecule or molecular complex that is sufficiently structurally homologous to a portion of S. aureus thioredoxin reductase can be resolved by this method. In addition to a molecule that shares one or more structural features with S. aureus thioredoxin reductase as described above, a molecule that has similar bioactivity, such as the same catalytic activity, substrate specificity or ligand binding activity as S. aureus thioredoxin reductase, may also be sufficiently structurally homologous to S. aureus thioredoxin reductase to permit use of the structure coordinates of S. aureus thioredoxin reductase to solve its crystal structure.

In a preferred embodiment, the method of molecular replacement is utilized to obtain structural information about a molecule or molecular complex, wherein the molecule or molecular complex comprises at least one S. aureus thioredoxin reductase subunit or homolog. A “subunit” of S. aureus thioredoxin reductase is an S. aureus thioredoxin reductase molecule that has been truncated at the N-terminus or the C-terminus, or both. In the context of the present invention, a “homolog” of S. aureus thioredoxin reductase is a protein that contains one or more amino acid substitutions, deletions, additions, or rearrangements with respect to the amino acid sequence of S. aureus thioredoxin reductase, but that, when folded into its native conformation, exhibits or is reasonably expected to exhibit at least a portion of the tertiary (three-dimensional) structure of S. aureus thioredoxin reductase. For example, structurally homologous molecules can contain deletions or additions of one or more contiguous or noncontiguous amino acids, such as a loop or a domain. Structurally homologous molecules also include “modified” S. aureus thioredoxin reductase molecules that have been chemically or enzymatically derivatized at one or more constituent amino acid, including side chain modifications, backbone modifications, and N- and C-terminal modifications including acetylation, hydroxylation, methylation, amidation, and the attachment of carbohydrate or lipid moieties, cofactors, and the like.

A heavy atom derivative of S. aureus thioredoxin reductase is also included as an S. aureus thioredoxin reductase homolog. The term “heavy atom derivative” refers to derivatives of S. aureus thioredoxin reductase produced by chemically modifying a crystal of S. aureus thioredoxin reductase. In practice, a crystal is soaked in a solution containing heavy metal atom salts, or organometallic compounds, e.g., lead chloride, gold thiomalate, thiomersal or uranyl acetate, which can diffuse through the crystal and bind to the surface of the protein. The location(s) of the bound heavy metal atom(s) can be determined by x-ray diffraction analysis of the soaked crystal. This information, in turn, is used to generate the phase information used to construct three-dimensional structure of the protein (T. L. Blundell and N. L. Johnson, Protein Crystallography, Academic Press (1976)).

Because S. aureus thioredoxin reductase can crystallize in more than one crystal form, the structure coordinates of S. aureus thioredoxin reductase as provided by this invention are particularly useful in solving the structure of other crystal forms of S. aureus thioredoxin reductase or S. aureus thioredoxin reductase complexes.

The structure coordinates of S. aureus thioredoxin reductase in Table 1 are also particularly useful to solve the structure of crystals of S. aureus thioredoxin reductase, S. aureus thioredoxin reductase mutants or S. aureus thioredoxin reductase homologs co-complexed with a variety of chemical entities. This approach enables the determination of the optimal sites for interaction between chemical entities, including candidate S. aureus thioredoxin reductase inhibitors and S. aureus thioredoxin reductase. Potential sites for modification within the various binding site of the molecule can also be identified. This information provides an additional tool for determining the most efficient binding interactions, for example, increased hydrophobic interactions, between S. aureus thioredoxin reductase and a chemical entity. For example, high resolution x-ray diffraction data collected from crystals exposed to different types of solvent allows the determination of where each type of solvent molecule resides. Small molecules that bind tightly to those sites can then be designed and synthesized and tested for their S. aureus thioredoxin reductase inhibition activity.

All of the complexes referred to above may be studied using well-known x-ray diffraction techniques and may be refined versus 1.5-3 Å resolution x-ray data to an R value of about 0.20 or less using computer software, such as X-PLOR (Yale University, 81992, distributed by Molecular Simulations, Inc.; see, e.g., Blundell & Johnson, supra; Meth. Enzymol., Vol. 114 & 115, H. W. Wyckoff et al., eds., Academic Press (1985)). This information may thus be used to optimize known S. aureus thioredoxin reductase inhibitors, and more importantly, to design new S. aureus thioredoxin reductase inhibitors.

The invention also includes the unique three-dimensional configuration defined by a set of points defined by the structure coordinates for a molecule or molecular complex structurally homologous to S. aureus thioredoxin reductase as determined using the method of the present invention, structurally equivalent configurations, and magnetic storage media comprising such set of structure coordinates.

Further, the invention includes structurally homologous molecules as identified using the method of the invention.

Homology Modeling

Using homology modeling, a computer model of an S. aureus thioredoxin reductase homolog can be built or refined without crystallizing the homolog. First, a preliminary model of the S. aureus thioredoxin reductase homolog is created by sequence alignment with S. aureus thioredoxin reductase, secondary structure prediction, the screening of structural libraries, or any combination of those techniques. Computational software may be used to carry out the sequence alignments and the secondary structure predictions. Structural incoherences, e.g., structural fragments around insertions and deletions, can be modeled by screening a structural library for peptides of the desired length and with a suitable conformation. For prediction of the side chain conformation, a side chain rotamer library may be employed. Where the S. aureus thioredoxin reductase homolog has been crystallized, the final homology model can be used to solve the crystal structure of the homolog by molecular replacement, as described above. Next, the preliminary model is subjected to energy minimization to yield an energy minimized model. The energy minimized model may contain regions where stereochemistry restraints are violated, in which case such regions are remodeled to obtain a final homology model. The homology model is positioned according to the results of molecular replacement, and subjected to further refinement comprising molecular dynamics calculations.

Rational Drug Design

Computational techniques can be used to screen, identify, select and design chemical entities capable of associating with S. aureus thioredoxin reductase or structurally homologous molecules. Knowledge of the structure coordinates for S. aureus thioredoxin reductase permits the design and/or identification of synthetic compounds and/or other molecules which have a shape complementary to the conformation of a binding site or active site of S. aureus thioredoxin reductase. In particular, computational techniques can be used to identify or design chemical entities, such as inhibitors, agonists and antagonists, that associate with a binding site or active site of S. aureus thioredoxin reductase or an S. aureus thioredoxin reductase-like binding site or active site. Inhibitors may bind to or interfere with all or a portion of the binding site or active site of S. aureus thioredoxin reductase, and can be competitive, non-competitive, or uncompetitive inhibitors; or interfere with dimerization by binding at the interface between the two monomers. Once identified and screened for biological activity, these inhibitors/agonists/antagonists may be used therapeutically or prophylactically to block S. aureus thioredoxin reductase activity and, thus, result in inhibition of growth or death of the bacteria. Structure-activity data for analogs of ligands that bind to or interfere with S. aureus thioredoxin reductase or S. aureus thioredoxin reductase-like binding sites can also be obtained computationally.

The term “chemical entity,” as used herein, refers to chemical compounds, complexes of two or more chemical compounds, and fragments of such compounds or complexes. Chemical entities that are determined to associate with S. aureus thioredoxin reductase are potential drug candidates. Data stored in a machine-readable storage medium that is capable of displaying a graphical three-dimensional representation of the structure of S. aureus thioredoxin reductase or a structurally homologous molecule, as identified herein, or portions thereof may thus be advantageously used for drug discovery. The structure coordinates of the chemical entity are used to generate a three-dimensional image that can be computationally fit to the three-dimensional image of S. aureus thioredoxin reductase or a structurally homologous molecule. The three-dimensional molecular structure encoded by the data in the data storage medium can then be computationally evaluated for its ability to associate with chemical entities. When the molecular structures encoded by the data is displayed in a graphical three-dimensional representation on a computer screen, the protein structure can also be visually inspected for potential association with chemical entities.

One embodiment of the method of drug design involves evaluating the potential association of a known chemical entity with S. aureus thioredoxin reductase or a structurally homologous molecule, particularly with an S. aureus thioredoxin reductase binding site or S. aureus thioredoxin reductase-like binding site. The method of drug design thus includes computationally evaluating the potential of a selected chemical entity to associate with any of the molecules or molecular complexes set forth above. This method comprises the steps of: (a) employing computational means to perform a fitting operation between the selected chemical entity and a binding site of the molecule or molecular complex; and (b) analyzing the results of said fitting operation to quantify the association between the chemical entity and the binding site.

In another embodiment, the method of drug design involves computer-assisted design of chemical entities that associate with S. aureus thioredoxin reductase, its homologs, or portions thereof. Chemical entities can be designed in a step-wise fashion, one fragment at a time, or may be designed as a whole or “de novo.”

To be a viable drug candidate, the chemical entity identified or designed according to the method must be capable of structurally associating with at least part of an S. aureus thioredoxin reductase or S. aureus thioredoxin reductase-like binding sites, and must be able, sterically and energetically, to assume a conformation that allows it to associate with the S. aureus thioredoxin reductase or S. aureus thioredoxin reductase-like binding site. Non-covalent molecular interactions important in this association include hydrogen bonding, van der Waals interactions, hydrophobic interactions, and electrostatic interactions. Conformational considerations include the overall three-dimensional structure and orientation of the chemical entity in relation to the binding site, and the spacing between various functional groups of an entity that directly interact with the S. aureus thioredoxin reductase-like binding site or homologs thereof.

Optionally, the potential binding of a chemical entity to an S. aureus thioredoxin reductase or S. aureus thioredoxin reductase-like binding site is analyzed using computer modeling techniques prior to the actual synthesis and testing of the chemical entity. If these computational experiments suggest insufficient interaction and association between it and the S. aureus thioredoxin reductase or S. aureus thioredoxin reductase-like binding site, testing of the entity is obviated. However, if computer modeling indicates a strong interaction, the molecule may then be synthesized and tested for its ability to bind to or interfere with an S. aureus thioredoxin reductase or S. aureus thioredoxin reductase-like binding site. Binding assays to determine if a compound actually binds to S. aureus thioredoxin reductase can also be performed and are well known in the art. Binding assays may employ kinetic or thermodynamic methodology using a wide variety of techniques including, but not limited to, microcalorimetry, circular dichroism, capillary zone electrophoresis, nuclear magnetic resonance spectroscopy, fluorescence spectroscopy, and combinations thereof.

One skilled in the art may use one of several methods to screen chemical entities or fragments for their ability to associate with an S. aureus thioredoxin reductase or S. aureus thioredoxin reductase-like binding site. This process may begin by visual inspection of, for example, an S. aureus thioredoxin reductase or S. aureus thioredoxin reductase-like binding site on the computer screen based on the S. aureus thioredoxin reductase structure coordinates in Table 1 or other coordinates which define a similar shape generated from the machine-readable storage medium. Selected fragments or chemical entities may then be positioned in a variety of orientations, or docked, within the binding site. Docking may be accomplished using software such as QUANTA and SYBYL, followed by energy minimization and molecular dynamics with standard molecular mechanics forcefields, such as CHARMM and AMBER.

Specialized computer programs may also assist in the process of selecting fragments or chemical entities. Examples include GRID (P. J. Goodford, J. Med. Chem. 28:849-857 (1985); available from Oxford University, Oxford, UK); MCSS (A. Miranker et al., Proteins: Struct. Funct. Gen., 11:29-34 (1991); available from Molecular Simulations, San Diego, Calif.); AUTODOCK (D. S. Goodsell et al., Proteins: Struct. Funct. Genet. 8:195-202 (1990); available from Scripps Research Institute, La Jolla, Calif.); and DOCK (I. D. Kuntz et al., J. Mol. Biol. 161:269-288 (1982); available from University of California, San Francisco, Calif.).

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of S. aureus thioredoxin reductase. This would be followed by manual model building using software such as QUANTA or SYBYL (Tripos Associates, St. Louis, Mo.).

Useful programs to aid one of skill in the art in connecting the individual chemical entities or fragments include, without limitation, CAVEAT (P. A. Bartlett et al., in Molecular Recognition in Chemical and Biological Problems,” Special Publ., Royal Chem. Soc., 78:182-196 (1989); G. Lauri et al., J. Comput. Aided Mol. Des., 8:51-66 (1994); available from the University of California, Berkeley, Calif.); 3D database systems such as ISIS (available from MDL Information Systems, San Leandro, Calif.; reviewed in Y. C. Martin, J. Med. Chem., 35:2145-2154 (1992)); and HOOK (M. B. Eisen et al., Proteins: Struc., Funct., Genet. 19:199-221 (1994); available from Molecular Simulations, San Diego, Calif.).

S. aureus thioredoxin reductase binding compounds may be designed “de novo” using either an empty binding site or optionally including some portion(s) of a known inhibitor(s). There are many de novo ligand design methods including, without limitation, LUDI (H.-J. Bohm, J. Comp. Aid. Molec. Design, 6:61-78 (1992); available from Molecular Simulations Inc., San Diego, Calif.); LEGEND (Y. Nishibata et al., Tetrahedron, 47:8985 (1991); available from Molecular Simulations Inc., San Diego, Calif.); LeapFrog (available from Tripos Associates, St. Louis, Mo.); and SPROUT (V. Gillet et al., J. Comput. Aided Mol. Design, 7:127-153 (1993); available from the University of Leeds, UK).

Once a compound has been designed or selected by the above methods, the efficiency with which that entity may bind to or interfere with an S. aureus thioredoxin reductase or S. aureus thioredoxin reductase-like binding site may be tested and optimized by computational evaluation. For example, an effective S. aureus thioredoxin reductase or S. aureus thioredoxin reductase-like binding site inhibitor must preferably demonstrate a relatively small difference in energy between its bound and free states (i.e., a small deformation energy of binding). Thus, the most efficient S. aureus thioredoxin reductase or S. aureus thioredoxin reductase-like binding site inhibitors should preferably be designed with a deformation energy of binding of not greater than about 10 kcal/mole; more preferably, not greater than 7 kcal/mole. S. aureus thioredoxin reductase or S. aureus thioredoxin reductase-like binding site inhibitors may interact with the binding site in more than one conformation that is similar in overall binding energy. In those cases, the deformation energy of binding is taken to be the difference between the energy of the free entity and the average energy of the conformations observed when the inhibitor binds to the protein.

An entity designed or selected as binding to or interfering with an S. aureus thioredoxin reductase or S. aureus thioredoxin reductase-like binding site may be further computationally optimized so that in its bound state it would preferably lack repulsive electrostatic interaction with the target enzyme and with the surrounding water molecules. Such non-complementary electrostatic interactions include repulsive charge-charge, dipole-dipole, and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. 81995); AMBER, version 4.1 (P. A. Kollman, University of California at San Francisco, 81995); QUANTA/CHARMM (Molecular Simulations, Inc., San Diego, Calif. 81995); Insight II/Discover (Molecular Simulations, Inc., San Diego, Calif. 81995); DelPhi (Molecular Simulations, Inc., San Diego, Calif. 81995); and AMSOL (Quantum Chemistry Program Exchange, Indiana University). These programs may be implemented, for instance, using a Silicon Graphics workstation such as an Indigo² with “IMPACT” graphics. Other hardware systems and software packages will be known to those skilled in the art.

Another approach encompassed by this invention is the computational screening of small molecule databases for chemical entities or compounds that can bind in whole, or in part, to an S. aureus thioredoxin reductase or S. aureus thioredoxin reductase-like binding site. In this screening, the quality of fit of such entities to the binding site may be judged either by shape complementarity or by estimated interaction energy (E. C. Meng et al., J. Comp. Chem., 13, pp. 505-524 (1992)).

This invention also enables the development of chemical entities that can isomerize to short-lived reaction intermediates in the chemical reaction of a substrate or other compound that binds to or with S. aureus thioredoxin reductase. Time-dependent analysis of structural changes in S. aureus thioredoxin reductase during its interaction with other molecules is carried out. The reaction intermediates of S. aureus thioredoxin reductase can also be deduced from the reaction product in co-complex with S. aureus thioredoxin reductase. Such information is useful to design improved analogs of known S. aureus thioredoxin reductase inhibitors or to design novel classes of inhibitors based on the reaction intermediates of the S. aureus thioredoxin reductase and inhibitor co-complex. This provides a novel route for designing S. aureus thioredoxin reductase inhibitors with both high specificity and stability.

Yet another approach to rational drug design involves probing the S. aureus thioredoxin reductase crystal of the invention with molecules comprising a variety of different functional groups to determine optimal sites for interaction between candidate S. aureus thioredoxin reductase inhibitors and the protein. For example, high resolution x-ray diffraction data collected from crystals soaked in or co-crystallized with other molecules allows the determination of where each type of solvent molecule sticks. Molecules that bind tightly to those sites can then be further modified and synthesized and tested for their hepes protease inhibitor activity (J. Travis, Science, 262:1374 (1993)).

In a related approach, iterative drug design is used to identify inhibitors of S. aureus thioredoxin reductase. Iterative drug design is a method for optimizing associations between a protein and a compound by determining and evaluating the three-dimensional structures of successive sets of protein/compound complexes. In iterative drug design, crystals of a series of protein/compound complexes are obtained and then the three-dimensional structures of each complex is solved. Such an approach provides insight into the association between the proteins and compounds of each complex. This is accomplished by selecting compounds with inhibitory activity, obtaining crystals of this new protein/compound complex, solving the three dimensional structure of the complex, and comparing the associations between the new protein/compound complex and previously solved protein/compound complexes. By observing how changes in the compound affected the protein/compound associations, these associations may be optimized.

Pharmaceutical Compositions

Pharmaceutical compositions of this invention comprise an inhibitor of S. aureus thioredoxin reductase activity identified according to the invention, or a pharmaceutically acceptable salt thereof, and a pharmaceutically acceptable carrier, adjuvant, or vehicle. The term “pharmaceutically acceptable carrier” refers to a carrier(s) that is “acceptable” in the sense of being compatible with the other ingredients of a composition and not deleterious to the recipient thereof. Optionally, the pH of the formulation is adjusted with pharmaceutically acceptable acids, bases, or buffers to enhance the stability of the formulated compound or its delivery form.

Methods of making and using such pharmaceutical compositions are also included in the invention. The pharmaceutical compositions of the invention can be administered orally, parenterally, by inhalation spray, topically, rectally, nasally, buccally, vaginally, or via an implanted reservoir. Oral administration or administration by injection is preferred. The term parenteral as used herein includes subcutaneous, intracutaneous, intravenous, intramuscular, intra-articular, intrasynovial, intrastemal, intrathecal, intralesional, and intracranial injection or infusion techniques.

Dosage levels of between about 0.01 and about 100 mg/kg body weight per day, preferably between about 0.5 and about 75 mg/kg body weight per day of the S. aureus thioredoxin reductase inhibitory compounds described herein are useful for the prevention and treatment of S. aureus thioredoxin reductase mediated disease. Typically, the pharmaceutical compositions of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

In order that this invention be more fully understood, the following examples are set forth. These examples are for the purpose of illustration only and are not to be construed as limiting the scope of the invention in any way.

### EXAMPLES

### Example 1

Analysis of the Structure of S. aureus Thioredoxin Reductase

Expression and Purification

S. aureus thioredoxin reductase was expressed from TU577 (M15 pQE60-TrxB), an expression strain constructed by GH Choi of Human Genome Sciences. A single colony was picked from a fresh streak plate into NS86 seed medium, grown to ˜1 A₅₅₀ and frozen ampoules (20% glycerol was added as a cryoprotectant) prepared. Ampoules were stored in the vapor phase of liquid nitrogen.

To prepare the seed, cells were grown in NS86 medium (2.6 g/L K₂HPO₄, 10.9 g/L NaNH4HPO₄.4H₂O, 2.1 g/L citric acid, 0.67 g/L (NH4)2SO₄, 0.25 g/L MgSO₄.7H₂O, 10.4 g/L yeast extract and 5 g/L glycerol) containing both ampicillin (100 micrograms/mL) and kanamycin (25 micrograms/mL). Shake flask medium was MIM (32 g/L tryptone, 20 g/L yeast extract, 6 g/L Na₂HPO₄, 3 g/L KH₂PO₄, 0.5 g/L NaCl, and 1 g/L NH₄Cl) containing ampicillin and kanamycin (100 and 25 micrograms/mL, respectively).

Seeds were prepared by the inoculation of 0.1 mL thawed ampoule contents into 50 mL of NS86 medium and grown overnight at 30° C. Flasks (4000 mL volume) containing 750 mL MIM medium were inoculated at 0.1 A₅₅₀. Cells were grown at 30° C., induced at a density of 1 A₅₅₀ by the addition of 1 mM IPTG and harvested at 2.5 hours post-induction by centrifugation.

For preparation of the selenomethionine analog of thioredoxin reductase, the construct was grown in minimal salts medium M9. Endogenous methionine biosynthesis was inhibited while adding an excess of selenomethionine to the growth medium just prior to induction of expression (Van Duyne et al., J. Mol. Biol., 229:105-24 (1993); and Benson et al., Nat. Struct. Biol., 2:644-53 (1995)). The formulation of basal M9 was Na₂HPO₄, 6 g; KH₂PO₄, 3 g; NH₄Cl, 1.0 g; and NaCl, 0.5 g per L of deionzed water. The pH was adjusted to 7.4 with concentrated KOH and the medium was sterilized by autoclaving. Prior to inoculation, the following filter sterilized solutions were added per L of basal medium: 1 M MgSO₄, 1.0 mL; 1 M CaCl₂, 0.1 mL; trace metal salts solution, 0.1 mL, 10 mM thiamin, 1.0 mL; and 20% glucose, 20 mL. The trace metal salts solution contained per L of deionized water: MgCl₂.6H₂O, 39.44 g; MnSO₄.H₂O, 5.58 g; FeSO₄.7H₂O, 1.11 g; Na₂MoO₄.2H2O, 0.48 g; CaCl₂, 0.33 g; NaCl, 0.12 g; and ascorbic acid, 1.0 g. Filter sterilized ampicillin and kanamycin were added to the medium at final concentrations of 100 micrograms/mL and 30 micrograms/mL, respectively.

Fermentations were prepared in 100 mL volumes of M9 medium contained in 500 mL wide mouth flasks. A 0.1 mL aliquot of the stock culture was inoculated into the medium and allowed to grow at 37° C. for 18-20 hours at 200 rpm. The seed culture was harvested by centrifugation and then resuspended in an equal volume of M9 medium. The resuspended seed was used to inoculate expression fermentations at a rate of 3%. For expression, the culture was grown under the same conditions to an A₆₀₀ of 0.6. At this point, methionine biosynthesis was down regulated by the addition of 6 amino acids. L-lysine, L-threonine, and L-phenylalanine were added to final concentrations of 100 micrograms/mL and L-leucine, L-isoleucine, and L-valine were added to 50 micrograms/mL. D,L-selenomethionine was added simultaneously to a final concentration of 100 micrograms/mL. After 15-20 minutes, protein expression was induced by addition of IPTG (isopropyl thio-β-D-galactosidase, Gibco BRL) to 1 mM. Growth of the culture was continued for an additional 3 hours until an A₆₀₀ of 1.5-1.6. Cells were then harvested by centrifugation and frozen at −80° C.

Cell paste was suspended in lysis buffer [50 mM Tris, pH 8.5, 5 mM β-mercaptoethanol, 1.8 g/L lysozyme, DNAse (either 100 mg/L of DNAse I from Sigma Chemical Co. (St. Louis, Mo.) or 20,000 Kunitz units of DNAse from Boehringer Manheim (Germany)), Complete™ protease inhibitor tablets (1 tablet per 50 ml, Boehringer Manheim)]. Cell suspensions were kept on ice for ˜1 hour, tissuemizing every 15-20 minutes. At the end of an hour, NaCl was added to each lysate to a final concentration of 0.5 M. Cell lysates were then clarified by centrifugation at 17,211×g (12,000 rpm, SS34 rotor) at 4° C. for ≧30 minutes.

Clarified cell lysates were loaded onto a 50 mL column (2.5 cm id) of Ni⁺²-NTA agarose (Qiagen, Inc., Valencia, Calif.) which had been pre-washed with water and equilibrated with buffer (50 mM Tris, 0.5 M NaCl, pH 8.5, 5 mM β-mercaptoethanol). In each experiment, the lysate was loaded onto the column, and then the column was washed with additional buffer until the absorbance returned to baseline. The column was eluted by washing with buffer containing 25 mM imidazole, 50 mM imidazole and then with buffer containing 100 mM imidazole. During the last two washes, the eluate was collected in 1 minute fractions (˜2.5 mL). Throughout the chromatography, the flow rate was 2.5 mL/min; the absorbance was monitored at 278 nm. Fractions were assayed for purity of thioredoxin reductase by sodium dodecylsulfate polyacrylamide-gel electrophoresis (SDS-PAGE).

S. aureus thioredoxin reductase purified by immobilized-metal affinity chromatography (IMAC) was dialyzed at 4° C. against 50 mM Tris, pH 8.0, 5 mM β-mercaptoethanol and loaded onto a 20 mL column (2.5 cm id) of Source Q (Amersham Pharmacia Biotech, Inc.; Piscataway, N.J.) which had been equilibrated in the same buffer. Following load, the column was washed with buffer and then eluted via a linear gradient from buffer to buffer+250 mM NaCl over 10 column volumes. Again, the column was washed and eluted at 2.5 mL/min; the eluate was monitored at 278 nm, and the absorbance was recorded at 2 and 0.2 AUFS. Fractions were assayed for purity of thioredoxin reductase by SDS-PAGE.

A pool of thioredoxin reductase from anion exchange chromatography was concentrated approximately 10-fold in a stirred cell with a YM3, 43 mm membrane. The concentrate was then loaded onto a 2.5 cm×96 cm column of Sephacryl S-100 (Amersham Pharmacia Biotech, Inc.; Piscataway, N.J.) which had been pre-equilibrated with 50 mM Tris, 1 mM EDTA, pH 8.0. Following load, the column was washed with >1 column volume of the same buffer. Throughout chromatography, absorbance was monitored at 278 nm and recorded at 2 AUFS and 0.1 mm/min. Fractions (3 mL) were assayed for purity of thioredoxin reductase by SDS-PAGE.

Crystallization/Structure Determination Overview

Purified S. aureus thioredoxin reductase was screened for crystallization conditions using Hampton Crystal Screen I and II (available from Hampton Research, Laguna Niguel, Calif.) and Wizard Screen I and II (available from Emerald Biostructures, Inc., Bainbridge Island, Wash.). Several hits were obtained in first round screening that were comprised of small yellow crystals. The most promising condition, Hampton Screen I condition number 33 (4M Sodium Formate), was explored using a follow-up screen to test the effect of decreasing concentrations of sodium formate on crystal formation. Optimal crystal growth was obtained between 3.2M and 3.6M sodium formate. The size and reproducibility of crystal formation was enhanced by elimination of the nonspecific protein precipitate and by streak seeding as described in the Materials and Methods. Selenomethionine S. aureus thioredoxin reductase was also prepared and crystallized to facilitate structure determination. A fluorescence scan near the K edge of selenium for the selenomethionine S. aureus thioredoxin reductase was recorded.

The structure was solved by multiple anomalous dispersion using the selenomethionine incorporated protein crystals. Two MAD data sets were collected at the Advanced Photon Source (Argonne, Ill.)—first data set (collected on the bending magnet beamline at station 17 (17-BM)) and the second data set (collected on the insertion device beamline at station 17 (17-ID)). The data collection statistics are given in Tables 8 and 9.

TABLE 8
Data collection and phasing statistics for the first data set
collected on the Bruker CCD detector on beamline 17-BM with an
exposure time of 30 sec and a frame width of 0.2°.

| | λ 0.95373Å | λ 0.97949Å | λ 0.97930Å |
|---|---|---|---|
| | (13000.0 eV) | (12658.0 eV) | (12660.5 eV) |
| Resolution | 2.3Å | 2.3Å | 2.3Å |
| Redundancy | 4.4 | 8.67 | 8.95 |
| No. unique refl. | 27,661 | 27,724 | 27,718 |
| % completeness | 95.5% | 95.7% | 95.7% |
| R_{sym} | 0.069 | 0.122 | 0.131 |
| R_{cullis} acentrics | — | 0.83 | 0.84 |
| R_{cullis} anomalous | — | 0.81 | 0.85 |
| Phasing power | | | |
| centrics | — | 0.80 | 0.75 |
| acentrics | — | 1.22 | 1.19 |

| | |
|---|---|
| Mean figure of merit (to 2.5Å resolution) | |
| before solvent flattening | 0.481 |
| after solvent flattening | 0.618 |

TABLE 9
Data collection and phasing statistics for the second data set
collected on the Mar CCD detector on beamline 17-ID with an
exposure time of 3 sec and a frame width of 0.5°.

| | λ 1.00000Å | λ 0.97953Å | λ 0.97939Å (12 |
|---|---|---|---|
| Resolution | 2.3Å | 2.3Å | 2.3Å |
| No. observations | 583,231 | 450,538 | 457,519 |
| No. unique refl. | 36,354 | 36,492 | 36,783 |
| % completeness | 99.9% | 99.9% | 99.6% |
| R_{sym} | 0.062 | 0.088 | 0.079 |
| R_{cullis} acentrics | — | 0.72 | 0.76 |
| R_{cullis} anomalous | — | 0.56 | 0.63 |
| Phasing power | | | |
| centrics | — | 1.16 | 0.93 |
| acentrics | — | 1.55 | 1.31 |

| | |
|---|---|
| Mean figure of merit | |
| (to 2.3Å resolution) | |
| before solvent flattening | 0.644 |
| after solvent flattening | 0.747 |

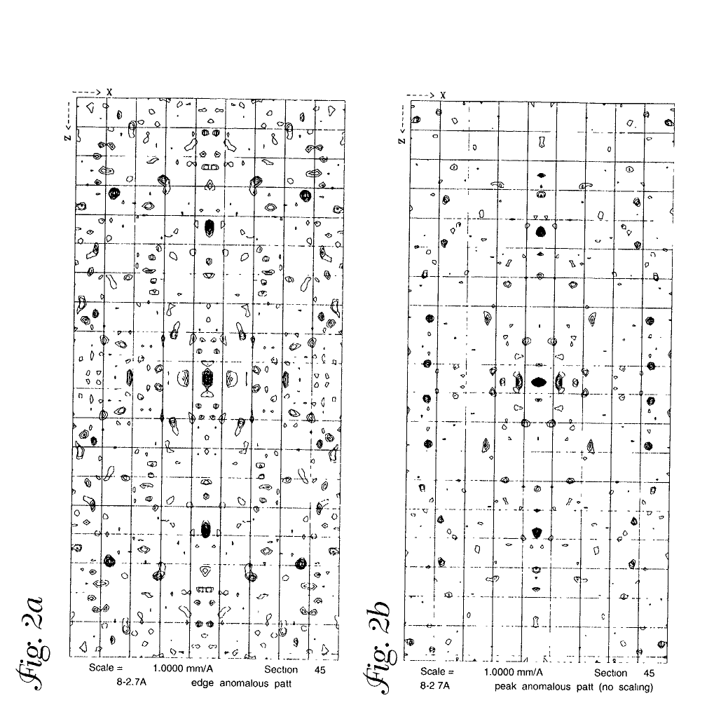

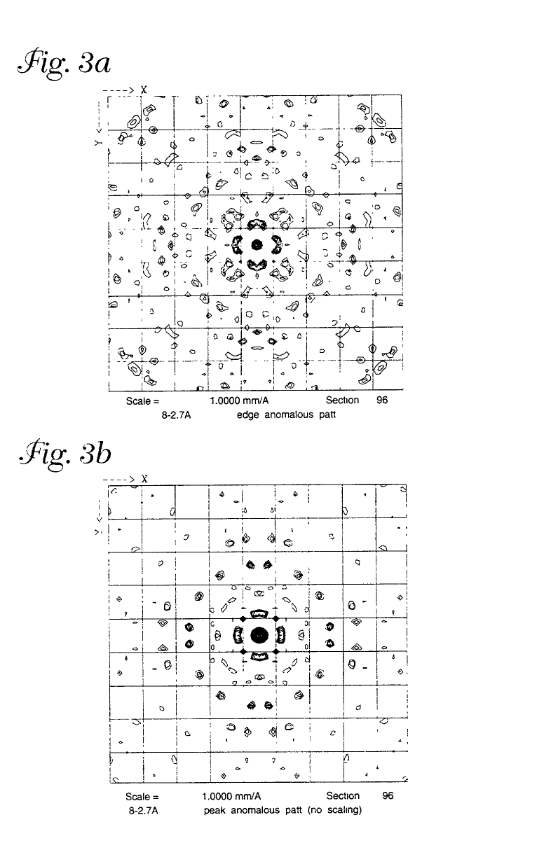

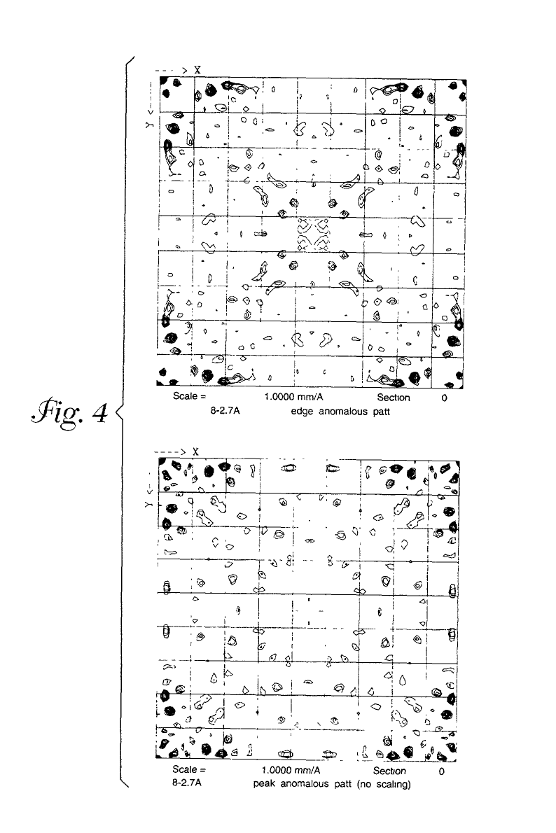

These two data sets were collected in order to test the quality of anomalous data collection on the bending magnet beamline which was undergoing validation. A precise comparison of these two experiments is limited by the fact that two different detectors were used to collect the data due to operational constraints (Mar CCD on 17-ID and Bruker CCD on 17-BM) and the intensity of X-rays was significantly weaker on 17-BM at the time of the experiment. A comparison of the anomalous difference Pattersons in FIGS. 2-4 and phasing statistics for the two data sets (Tables 8 and 9) indicate that while both data sets provided sufficient phase information to solve the structure, the data from the second data set was of higher quality. Therefore, the second data set, which includes data to 2.3 Å resolution, was used for the structure solution and refinement described herein. The crystals used for the second data set had unit cell constants of a=b=90.5 Å, c=193.2 Å, α=β=γ=90° in space group P4₃2₁2. There are two thioredoxin reductase molecules per asymmetric unit resulting in a solvent content of 55%. This is the first reported thioredoxin reductase crystal structure where the asymmetric unit is the functional dimer as opposed to the dimer resulting from crystallographic symmetry.

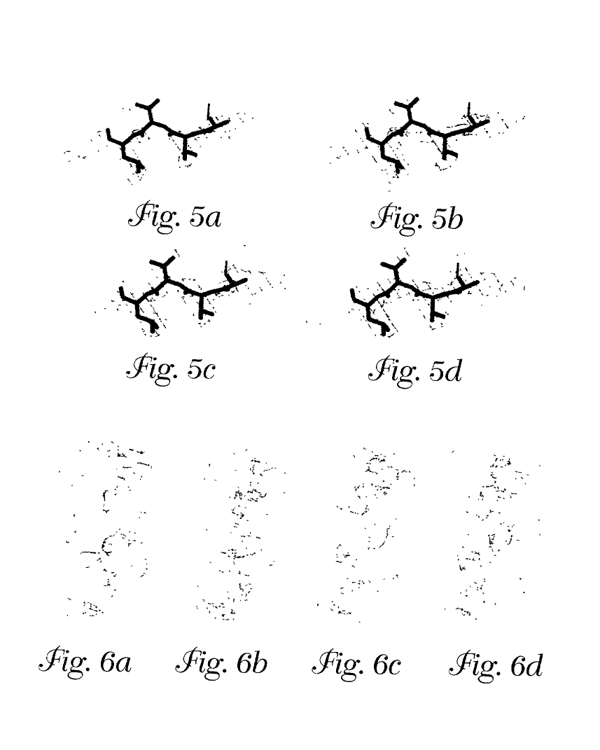

The selenomethionine structure solution required the identification of the positions of 14 selenium atoms. There are seven methionines per monomer excluding the N-terminal Met which is usually disordered. Anomalous difference Patterson maps revealed 8 selenium sites whose locations were readily derived using the automated Patterson search algorithm in SHELX (Sheldrick et al., Acta Cryst., B51:423-31 (1995)). Four of the eight sites were able to identify the other four sites using anomalous difference Fourier methods and vice versa. Three additional sites were identified in each of the anomalous difference Fourier maps phased with each of the two sets of four sites found by Patterson methods. These three new sites were used for phasing and able to successfully identify the initial eight sites. The eleven sites were analyzed using a graphical display. A definitive two-fold axis was observed and suggested one additional site which did not have a symmetry mate. The last two sites were identified by phasing with the eleven sites and choosing peaks that should be related by non-crystallographic symmetry. One peak found via this method was a true methionine position, and non-crystallographic symmetry was used to identify the final site. Phasing statistics (Tables 8 and 9) and electron density maps (FIGS. 5 and 6) indicate that the multiple anomalous dispersion experiment was successful in producing suitable phases of high enough quality to permit a straightforward tracing of the chain. The structure was built in CHAIN (Sack, Journal of Molecular Graphics, 6:224-25 (1988)) using the E. coli thioredoxin reductase as a starting model and refined using X-PLOR (Brunger, X-PLOR version 3.1, Yale University Press (1992)) to an R-factor of 25.8% and a Free R-factor of 29.4%.

Crystallization

The sample was delivered in 50 mM Tris, 1 mM EDTA, pH 8.0 for crystallization screening. Protein concentration was measured using the absorbance at 454 nm. S. aureus thioredoxin reductase was concentrated to 15 mg/mL using a an Ultra-Free 4 concentrator with a 10,000 Da molecular weight cutoff available from Millipore (Bedford, Mass.). Initial screening for crystallization conditions was conducted using Hampton Crystal Screen I (available from Hampton Research, Laguna Niguel, Calif.) and Wizard Screen I (available from Emerald Biostructures, Inc., Bainbridge Island, Wash.). Crystals or microcrystals were obtained in conditions 4, 16, 29, 33, and 38 of Hampton Crystal Screen I and conditions 6, 14, 18, 29, 34, 36 of Wizard Screen I. Cryo Screens I and II (available from Emerald Biostructures, Inc., Bainbridge Island, Wash.) were also tested without success. A follow-up screen for Hampton Crystal Screen I condition 33 (4M sodium formate) was conducted by varying sodium formate and protein concentrations. Crystals grew over a period of 1 to 3 days. Cryogenic solution conditions were obtained by transferring the crystals to 4M sodium formate just prior to freezing.

Further optimization of the crystallization conditions included steps to eliminate nonspecific precipitated protein. A 10 mg/mL stock solution of thioredoxin reductase was mixed with an equal volume of with 4M sodium formate to give a final protein concentration of 5 mg/mL in 2M sodium formate. Incubation of the sample at room temperature for 1 hr led to formation of a light protein precipitate which was easily removed by running the sample through a spin filter. The protein/sodium formate solution was then placed on the pedestal of sitting drop trays without any further dilution. Equilibration with the well buffer (3.0-4.0 M sodium formate depending on the screen) continued overnight. The next day each sitting drop was streak seeded from a stock of S. aureus thioredoxin reductase microcrystals in 4M sodium formate using a cat whisker.

Structure Determination

Two selenomethionine multiple anomalous dispersion (MAD) experiments were performed (first data set, 2.5 Å resolution, and second data set, 2.3 Å resolution) using three different wavelengths (see Tables 8 and 9). Each of these individual data sets was indexed and integrated separately (see Tables 8 and 9 for integration statistics). The data sets for each experiment were scaled to each other using the program SCALEIT in the CCP4 Program Suite (Collaborative Computational Project N4, Acta Cryst., D50:760-63 (1994)). Patterson maps revealed eight selenium sites whose locations were determined by direct methods using SHELX (Sheldrick et al., Acta Cryst., B51:423-31 (1995)). Data from the second data set showed the strongest anomalous signal and was used for phase determination. Two pairs of four sites were tested for authenticity by their ability to generate phases which could identify the other pair of sites in anomalous difference Fourier calculations. Subsequent sites were identified by iterative anomalous difference Fourier methods as described in the text. All heavy atom parameter refinement and phasing calculations were carried out with MLPHARE (Otwinowski, Isomorphous Replacement and Anomalous Scattering, W. Wolf, P. R. Evans, and A. G. W. Leslie, eds, 80-86 (1991); Collaborative Computational Project N4, Acta Cryst., D50:760-63 (1994)) by treating the remote wavelength as native and the edge and peak wavelengths as derivatives (Ramakrishnan et al., Nature, 362:219-23 (1993)). The phases were subsequently subjected to solvent flattening using the program DM (Cowtan et al., Acta Cryst., D49:148-57 (1993); Cowtan et al., Acta Cryst., D54:487-93 (1998); and Collaborative Computational Project N4, Acta Cryst., D50:760-63 (1994)).

Model building was done using the program CHAIN (Sack, Journal of Molecular Graphics, 6:224-25 (1988)) and LORE (Finzel, Meth. Enzymol., 277:230-42 (1997)). Model for one monomer built using the E. coli thioredoxin reductase structure as a reference. The second monomer placed using non-crystallographic symmetry (R-factor/Free R-factor=49.6%/49.8%), and its orientation was refined using rigid body rotation (R-factor/Free R-factor=43.7%/45.0%). One cycle of positional refinement, torision angle dynamics refinement, and individual B factor refinement led to significant improvement in the model (R-factor/Free R-factor=31.2%/137.9%). At this stage waters were added and each monomer was thoroughly checked against the electron density. A second round of refinement led to the present model (R-factor/Free R-factor=25.8%/29.4%, Table 10).

TABLE 10
Refinement Statistics for Data Set One

| | R-factor | Free R-factor | No. of reflections |
|---|---|---|---|
| 20-2.3Å F≧2σ | 0.2583 | 0.2937 | 34,352 |

| | Bonds (Å) | Angles (°) |
|---|---|---|
| r.m.s deviation from ideal geometry | 0.008 | 1.38 |

| | Number of atoms | Average B-factor |
|---|---|---|
| Protein | 4635 | 29.7 |
| Waters | 157 | 40.2 |
| Cofactors | 106 | 27.5 |
| Total | 4897 | 30.0 |

All refinement cycles were carried out with XPLOR98 (Brunger, X-PLOR version 3.1, Yale University Press (1992)) incorporating bulk solvent correction during the refinement (Jiang et al., J. Mol. Biol., 243:100-15 (1994)). Progress of the refinement was monitored by a decrease in both the R-factor and Free R-factor. Stereochemistry of the model was checked using PROCHECK (Laskowski et al., J. Appl. Cryst., 26:283-91 (1993)) revealing no residues in disallowed regions of the Ramachandran plot. FIGS. 5-6 and 8 were made using SETOR (Evans, J. Mol. Graphics, 11:134-38 (1993)) and FIG. 7 was produced with both MOLSCRIPT (Kraulis, J. Appl. Cryst., 24:946-50 (1991)) and Raster 3D (Merritt 1994) while FIGS. 10, 12-14, and 16-17 were produced in MOLSCRIPT (Kraulis, J. Appl. Cryst., 24:946-50 (1991)) alone.

The complete disclosure of all patents, patent applications including provisional applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described; many variations will be apparent to one skilled in the art and are intended to be included within the invention defined by the claims.

### SEQUENCE LISTING FREE TEXT

SEQ ID NO: 1 recombinant S. aureus thioredoxin reductase (TrxB)

SEQ ID NO: 2 recombinant E. coli thioredoxin reductase (TrxB)

SEQ ID NO: 3 recombinant A. thaliana thioredoxin reductase (TrxB)

What is claimed is:

1. A molecule or molecular complex comprising at least a portion of an S. aureus thioredoxin reductase or thioredoxin reductase-like FAD binding site, wherein the FAD binding site comprises the amino acids listed in Table 2, the FAD binding site being defined by a set of points having a root mean square deviation of less than about 1.1 Å from points representing the backbone atoms of said amino acids as represented by the structure coordinates listed in Table 1.

2. The molecule or molecular complex of claim 1, wherein the FAD binding site comprises the amino acids listed in Table 3.

3. The molecule or molecular complex of claim 1, wherein the FAD binding site comprises the amino acids listed in Table 4.

4. A molecule or molecular complex comprising at least a portion of an S. aureus thioredoxin reductase or thioredoxin reductase-like NADPH binding site, wherein the NADPH binding site comprises Cys 135, Cys 138, and the amino acids listed in Table 5, the NADPH binding site being defined by a set of points having a root mean square deviation of less than about 0.8 Å from points representing the backbone atoms of said amino acids as represented by the structure coordinates listed in Table 1.

5. The molecule or molecular complex of claim 4, wherein the NADPH binding site comprises Cys 135, Cys 138, and the amino acids listed in Table 6.

6. The molecule or molecular complex of claim 4, wherein the NADPH binding site comprises Cys 135, Cys 138, and the amino acids listed in Table 7.

7. A molecule or molecular complex that is structurally homologous to an S. aureus thioredoxin reductase molecule or molecular complex, wherein the S. aureus thioredoxin reductase molecule or molecular complex is represented by at least a portion of the structure coordinates listed in Table 1.

8. A scalable three dimensional configuration of points, at least a portion of said points derived from structure coordinates of at least a portion of an S. aureus thioredoxin reductase molecule or molecular complex listed in Table 1 comprising at least one of a thioredoxin reductase or thioredoxin reductase-like FAD binding site or an NADPH binding site.

9. A scalable three dimensional configuration of points, wherein substantially all of said points are derived from structure coordinates of an S. aureus thioredoxin reductase molecule or molecular complex listed in Table 1.

10. The scalable three dimensional configuration of points of claim 8 wherein at least a portion of the points derived from the S. aureus thioredoxin reductase structure coordinates are derived from structure coordinates representing the locations of at least the backbone atoms of amino acids defining an S. aureus thioredoxin reductase FAD binding site, the FAD binding site comprising the amino acids listed in Table 2.

11. The scalable three dimensional configuration of points of claim 10 wherein the FAD binding site comprises the amino acids listed in Table 3.

12. The scalable three dimensional configuration of points of claim 10 wherein the FAD binding site comprises the amino acids listed in Table 4.

13. The scalable three dimensional configuration of points of claim 8 wherein at least a portion of the points derived from the S. aureus thioredoxin reductase structure coordinates are derived from structure coordinates representing the locations of at least the backbone atoms of amino acids defining an S. aureus thioredoxin reductase NADPH binding site, the NADPH binding site comprising Cys 135, Cys 138, and the amino acids listed in Table 5.

14. The scalable three dimensional configuration of points of claim 13 wherein the NADPH binding site comprises Cys 135, Cys 138, and the amino acids listed in Table 6.

15. The scalable three dimensional configuration of points of claim 13 wherein the NADPH binding site comprises Cys 135, Cys 138, and the amino acids listed in Table 7.

16. The scalable three dimensional configuration of points of claim 8 displayed as a holographic image, a stereodiagram, a model or a computer-displayed image.

17. A scalable three dimensional configuration of points, at least a portion of the points derived from structure coordinates of at least a portion of a molecule or a molecular complex that is structurally homologous to an S. aureus thioredoxin reductase molecule or molecular complex and comprises at least one of an S. aureus thioredoxin reductase or thioredoxin reductase-like FAD binding site or an NADPH binding site.

18. The scalable three-dimensional configuration of points of claim 17 displayed as a holographic image, a stereodiagram, a model or a computer-displayed image

19. A machine-readable data storage medium comprising a data storage material encoded with machine readable data which, when using a machine programmed with instructions for using said data, is capable of displaying a graphical three-dimensional representation of at least one molecule or molecular complex selected from the group consisting of:
(i) a molecule or molecular complex comprising at least a portion of an S. aureus thioredoxin reductase or thioredoxin reductase-like FAD binding site comprising the amino acids listed in Table 2, the FAD binding site defined by a set of points having a root mean square deviation of less than about 1.1 Å from points representing the backbone atoms of said amino acids as represented by structure coordinates listed in Table 1; and
(ii) a molecule or molecular complex comprising at least a portion of an S. aureus thioredoxin reductase or thioredoxin reductase-like NADPH binding site comprising Cys 135, Cys 138, and the amino acids listed in Table 5, the NADPH binding site defined by a set of points having a root mean square deviation of less than about 0.8 Å from points representing the backbone atoms of said amino acids as represented by structure coordinates listed in Table 1; and
(iii) a molecule or molecular complex that is structurally homologous to an S. aureus thioredoxin reductase molecule or molecular complex, wherein the S. aureus thioredoxin reductase molecule or molecular complex is represented by at least a portion of the structure coordinates listed in Table 1.

20. A machine-readable data storage medium comprising a data storage material encoded with a first set of machine readable data which, when combined with a second set of machine readable data, using a machine programmed with instructions for using said first set of data and said second set of data, can determine at least a portion of the structure coordinates corresponding to the second set of machine readable data, wherein said first set of data comprises a Fourier transform of at least a portion of the structural coordinates for S. aureus thioredoxin reductase listed in Table 1; and said second set of data comprises an x-ray diffraction pattern of a molecule or molecular complex of unknown structure.

21. A method for obtaining structural information about a molecule or a molecular complex of unknown structure comprising:
crystallizing the molecule or molecular complex;
generating an x-ray diffraction pattern from the crystallized molecule or molecular complex;
applying at least a portion of the structure coordinates set forth Table 1 to the x-ray diffraction pattern to generate a three-dimensional electron density map of at least a portion of the molecule or molecular complex whose structure is unknown.

22. A method for homology modeling an S. aureus thioredoxin reductase homolog comprising:
aligning the amino acid sequence of an S. aureus thioredoxin reductase homolog with an amino acid sequence of S. aureus thioredoxin reductase and incorporating the sequence of the S. aureus thioredoxin reductase homolog into a model of S. aureus thioredoxin reductase derived from structure coordinates set forth in Table 1 to yield a preliminary model of the S. aureus thioredoxin reductase homolog;
subjecting the preliminary model to energy minimization to yield an energy minimized model;
remodeling regions of the energy minimized model where stereochemistry restraints are violated to yield a final model of the S. aureus thioredoxin reductase homolog.

23. A computer-assisted method for identifying an inhibitor of S. aureus thioredoxin reductase activity comprising:
supplying a computer modeling application with a set of structure coordinates of a molecule or molecular complex, the molecule or molecular complex comprising at least a portion of an S. aureus thioredoxin reductase or thioredoxin reductase-like FAD binding site, the FAD binding site comprising the amino acids listed in Table 2;
supplying the computer modeling application with a set of structure coordinates of a chemical entity; and
determining whether the chemical entity is an inhibitor expected to bind to or interfere with the molecule or molecular complex, wherein binding to or interfering with the molecule or molecular complex is indicative of potential inhibition of S. aureus thioredoxin reductase activity.

24. A computer-assisted method for identifying an inhibitor of S. aureus thioredoxin reductase activity comprising:
supplying a computer modeling application with a set of structure coordinates of a molecule or molecular complex, the molecule or molecular complex comprising at least a portion of an S. aureus thioredoxin reductase or thioredoxin reductase-like NADPH binding site, the NADPH binding site comprising Cys 135, Cys 138, and the amino acids listed in Table 5;
supplying the computer modeling application with a set of structure coordinates of a chemical entity; and
determining whether the chemical entity is an inhibitor expected to bind to or interfere with the molecule or molecular complex, wherein binding to or interfering with the molecule or molecular complex is indicative of potential inhibition of S. aureus thioredoxin reductase activity.

25. The method of claim 23, wherein the FAD binding site comprises the amino acids listed in Table 2, the FAD binding site being defined by a set of points having a root mean square deviation of less than about 1.1 Å from points representing the backbone atoms of said amino acids as represented by structure coordinates listed in Table 1.

26. The method of claim 24, wherein the NADPH binding site comprises Cys 135, Cys 138, and the amino acids listed in Table 5, the NADPH binding site being defined by a set of points having a root mean square deviation of less than about 0.8 Å from points representing the backbone atoms of said amino acids as represented by structure coordinates listed in Table 1.

27. The method of claim 23 or 24, wherein determining whether the chemical entity is an inhibitor expected to bind to or interfere with the molecule or molecular complex comprises performing a fitting operation between the chemical entity and a binding site of the molecule or molecular complex, followed by computationally analyzing the results of the fitting operation to quantify the association between, or the interference with, the chemical entity and the binding site.

28. The method of claim 23 or 24 further comprising screening a library of chemical entities.

29. A computer-assisted method for designing an inhibitor of S. aureus thioredoxin reductase activity comprising:
supplying a computer modeling application with a set of structure coordinates of a molecule or molecular complex, the molecule or molecular complex comprising at least a portion of an S. aureus thioredoxin reductase or thioredoxin reductase-like FAD binding site, the FAD binding site comprising the amino acids listed in Table 2;
supplying the computer modeling application with a set of structure coordinates for a chemical entity;
evaluating the potential binding or interfering interactions between the chemical entity and the FAD binding site of the molecule or molecular complex;
structurally modifying the chemical entity to yield a set of structure coordinates for a modified chemical entity; and
determining whether the modified chemical entity is an inhibitor expected to bind to or interfere with the molecule or molecular complex, wherein binding to the molecule or molecular complex is indicative of potential inhibition of S. aureus thioredoxin reductase activity.

30. A computer-assisted method for designing an inhibitor of S. aureus thioredoxin reductase activity comprising:
supplying a computer modeling application with a set of structure coordinates of a molecule or molecular complex, the molecule or molecular complex comprising at least a portion of an S. aureus thioredoxin reductase or thioredoxin reductase-like NADPH binding site, the NADPH binding site comprising Cys 135, Cys 138, and the amino acids listed in Table 5;
supplying the computer modeling application with a set of structure coordinates for a chemical entity;
evaluating the potential binding or interfering interactions between the chemical entity and the NADPH binding site of the molecule or molecular complex;
structurally modifying the chemical entity to yield a set of structure coordinates for a modified chemical entity; and
determining whether the modified chemical entity is an inhibitor expected to bind to or interfere with the molecule or molecular complex, wherein binding to the molecule or molecular complex is indicative of potential inhibition of S. aureus thioredoxin reductase activity.

31. The method of claim 29, wherein the FAD binding site comprises the amino acids listed in Table 2, the FAD binding site being defined by a set of points having a root mean square deviation of less than about 1.1 Å from points representing the backbone atoms of said amino acids as represented by structure coordinates listed in Table 1.

32. The method of claim 30, wherein the NADPH binding site comprises Cys 135, Cys 138, and the amino acids listed in Table 5, the NADPH binding site being defined by a set of points having a root mean square deviation of less than about 0.8 Å from points representing the backbone atoms of said amino acids as represented by structure coordinates listed in Table 1.

33. The method of claim 29 or 30, wherein determining whether the modified chemical entity is an inhibitor expected to bind to or interfere with the molecule or molecular complex comprises performing a fitting operation between the chemical entity and a binding site of the molecule or molecular complex, followed by computationally analyzing the results of the fitting operation to quantify the association between or interference with the chemical entity and the binding site.

34. The method of claim 29 or 30, wherein the set of structure coordinates for the chemical entity is obtained from a chemical fragment library

35. A computer-assisted method for designing an inhibitor of S. aureus thioredoxin reductase activity de novo comprising:
supplying a computer modeling application with a set of structure coordinates of a molecule or molecular complex, the molecule or molecular complex comprising at least a portion of an S. aureus thioredoxin reductase or thioredoxin reductase-like FAD binding site, wherein the FAD binding site comprises the amino acids listed in Table 2;
computationally building a chemical entity represented by set of structure coordinates; and
determining whether the chemical entity is an inhibitor expected to bind to or interfere with the molecule or molecular complex, wherein binding to or interfering with the molecule or molecular complex is indicative of potential inhibition of S. aureus thioredoxin reductase activity.

36. A computer-assisted method for designing an inhibitor of S. aureus thioredoxin reductase activity de novo comprising:
supplying a computer modeling application with a set of structure coordinates of a molecule or molecular complex, the molecule or molecular complex comprising at least a portion of an S. aureus thioredoxin reductase NADPH binding site, wherein the NADPH binding site comprises Cys 135, Cys 138, and the amino acids listed in Table 5;
computationally building a chemical entity represented by set of structure coordinates; and
determining whether the chemical entity is an inhibitor expected to bind to or interfere with the molecule or molecular complex, wherein binding to or interfering with the molecule or molecular complex is indicative of potential inhibition of S. aureus thioredoxin reductase activity.

37. The method of claim 35, wherein the FAD binding site comprises the amino acids listed in Table 2, the FAD binding site being defined by a set of points having a root mean square deviation of less than about 1.1 Å from points representing the backbone atoms of said amino acids as represented by structure coordinates listed in Table 1.

38. The method of claim 36, wherein the NADPH binding site comprises Cys 135, Cys 138, and the amino acids listed in Table 5, the NADPH binding site being defined by a set of points having a root mean square deviation of less than about 0.8 Å from points representing the backbone atoms of said amino acids as represented by structure coordinates listed in Table 1.

39. The method of claim 35 or 36, wherein determining whether the chemical entity is an inhibitor expected to bind to or interfere with the molecule or molecular complex comprises performing a fitting operation between the chemical entity and a binding site of the molecule or molecular complex, followed by computationally analyzing the results of the fitting operation to quantify the association between or interference with the chemical entity and the binding site.

40. The method of any of claims 23, 24, 29, 30, 35, or 36 further comprising providing the potential inhibitor, then assaying the potential inhibitor to determine whether it inhibits S. aureus thioredoxin reductase activity.

41. A method for making an inhibitor of S. aureus thioredoxin reductase activity, the method comprising chemically or enzymatically synthesizing a chemical entity to yield an inhibitor of S. aureus thioredoxin reductase activity, the chemical entity having been identified during a computer-assisted process comprising supplying a computer modeling application with a set of structure coordinates of a molecule or molecular complex, the molecule or molecular complex comprising at least a portion of an S. aureus thioredoxin reductase or thioredoxin reductase-like FAD binding site or an NADPH binding site; supplying the computer modeling application with a set of structure coordinates of a chemical entity; and determining whether the chemical entity is expected to bind to or interfere with the molecule or molecular complex at a binding site, wherein binding to or interfering with the molecule or molecular complex is indicative of potential inhibition of S. aureus thioredoxin reductase activity.

42. A method for making an inhibitor of S. aureus thioredoxin reductase activity, the method comprising chemically or enzymatically synthesizing a chemical entity to yield an inhibitor of S. aureus thioredoxin reductase activity, the chemical entity having been designed during a computer-assisted process comprising supplying a computer modeling application with a set of structure coordinates of a molecule or molecular complex, the molecule or molecular complex comprising at least a portion of an S. aureus thioredoxin reductase or thioredoxin reductase-like FAD binding site or NADPH binding site; supplying the computer modeling application with a set of structure coordinates for a chemical entity; evaluating the potential binding interactions between the chemical entity and a binding site of the molecule or molecular complex; structurally modifying the chemical entity to yield a set of structure coordinates for a modified chemical entity; and determining whether the chemical entity is expected to bind to or interfere with the molecule or molecular complex at the binding site, wherein binding to or interfering with the molecule or molecular complex is indicative of potential inhibition of S. aureus thioredoxin reductase activity.

43. A method for making an inhibitor of S. aureus thioredoxin reductase activity, the method comprising chemically or enzymatically synthesizing a chemical entity to yield an inhibitor of S. aureus thioredoxin reductase activity, the chemical entity having been designed during a computer-assisted process comprising supplying a computer modeling application with a set of structure coordinates of a molecule or molecular complex, the molecule or molecular complex comprising at least a portion of an S. aureus thioredoxin reductase or thioredoxin reductase-like FAD binding site or an NADPH binding site; computationally building a chemical entity represented by set of structure coordinates; and determining whether the chemical entity is expected to bind to or interfere with the molecule or molecular complex at a binding site, wherein binding to or interfering with the molecule or molecular complex is indicative of potential inhibition of S. aureus thioredoxin reductase activity.

44. An inhibitor of S. aureus thioredoxin reductase activity identified, designed or made according to the method of any of the claims 23, 24, 29, 30, 35, 36, 41, 42, or 43.

45. A composition comprising an inhibitor of S. aureus thioredoxin reductase activity identified, designed or made according to the method of any of claims 23, 24, 29, 30, 35, 36, 41, 42, or 43.

46. A pharmaceutical composition comprising an inhibitor of S. aureus thioredoxin reductase activity identified or designed according to the method of any of claims 23, 24, 29, 30, 35, 36, 41, 42, or 43 or a salt thereof, and pharmaceutically acceptable carrier.

47. A method for crystallizing an S. aureus thioredoxin reductase molecule or molecular complex comprising:
preparing purified S. aureus thioredoxin reductase at a concentration of about 1 mg/ml to about 50 mg/ml; and
crystallizing S. aureus thioredoxin reductase from a solution at a pH of about 6 to about 10 and comprising about 0 wt. % to about 40 wt. % DMSO and about 100 mM to about 6 M sodium formate.

48. A crystal of S. aureus thioredoxin reductase.

49. The crystal of claim 48 having the tetragonal space group symmetry P4₃2₁2.

50. The crystal of claim 48 comprising a unit cell having dimensions a, b, and c; wherein a is about 70 Å to about 110 Å, b is about 70 Å to about 110 Å, c is about 160 Å to about 220 Å, and α=β=γ=90°.

51. The crystal of claim 48 comprising atoms arranged in a spatial relationship represented by the structure coordinates listed in Table 1.

52. The crystal of claim 48 wherein thioredoxin reductase has amino acid sequence SEQ ID NO:1.

53. The crystal of claim 48 wherein thioredoxin reductase amino acid sequence SEQ ID NO:1, except that at least one methionine is replaced with selenomethionine.
